SEQUENCE LISTING (2 sequences)

SEQ ID NO: 1 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

(19) United States
(12) Patent Application Publication
(10) Pub. No.: US 2026/0116976 A1
(43) Pub. Date: Apr. 30, 2026

(54) GENETICALLY DEFINED IMMUNE-CHECKPOINT INHIBITOR RESISTANCE IN AGGRESSIVE PRECURSORS OF HPV--HEAD AND NECK SQUAMOUS CANCER

(71) Applicants: THE REGENTS OF THE UNIVERSITY OF CALIFORNIA, Oakland, CA (US); DANA-FARBER CANCER INSTITUTE, INC., Boston, MA (US)

(72) Inventors: Scott M. Lippman, La Jolla, CA (US); Ludmil B. Alexandrov, San Diego, CA (US); Shuvro P. Nandi, San Diego, CA (US); Webster K. Cavenee, Del Mar, CA (US); Glenn J. Hanna, Boston, MA (US)

(21) Appl. No.: 19/116,947

(22) PCT Filed: Oct. 2, 2023
(86) PCT No.: PCT/US2023/075735

§ 371 Date: Mar. 28, 2025

Related U.S. Application Data
(60) Provisional application No. 63/412,835, filed on Oct. 3, 2022.

(51) Int. Cl. C07K 16/28; A61K 39/00; A61P 35/00; C12Q 1/6886

(52) U.S. Cl. CPC C07K 16/2818; A61P 35/00; C12Q 1/6886; A61K 2039/505; C12Q 2600/106; C12Q 2600/156

(57) ABSTRACT

Applicant provides herein methods for treating a patient having a pre-cancerous oral leukoplakia (LK) lesion or for delaying the progression of a pre-cancerous oral LK to oral squamous cell carcinoma. It also provides methods to identify patients that more likely to respond to a therapy comprising administration of an effective amount of an anti-PD-1 or anti-PD-L 1 therapy.

### CROSS-REFERENCE TO RELATED APPLICATION

This application claims priority under 35 U.S.C. § 119(e) to U.S. Provisional Ser. No. 63/412,835, filed Oct. 3, 2022, the contents of which are hereby incorporated by reference into the present disclosure in its entirety.

### BACKGROUND

Oral proliferative leukoplakia (PL) is an aggressive precancerous disease characterized by an extremely high-risk of transformation to oral squamous cell carcinoma (OSCC). Oral leukoplakia refers to a white plaque of questionable cancer risk having excluded other conditions and impacts up to 4% of the global population. There are no effective therapies that impact the natural history of malignant transformation. Thus, a need exists in the art to assess and treat PL, prior to transformation to oral squamous cell carcinoma.

### SUMMARY OF THE DISCLOSURE

Proliferative leukoplakia (PL) defines a distinct subgroup of aggressive leukoplakia with a high rate of malignant transformation approaching 10% per year. PL is characterized by heterogeneous, verrucous, or erythroleukoplakia lesions that typically involving multiple oral mucosal subsites. The 5-year cancer-free survival (CFS) for PL is estimated at 47%.

Oral leukoplakia refers to a white plaque of variable cancer risk having excluded other conditions and impacts up to 5% of the global population¹, but only a small proportion of leukoplakia lesions will undergo malignant transformation². Degree of epithelial dysplasia, lesion size, and tobacco history all influence the transformation rate³. Proliferative verrucous leukoplakia (PVL) defines an aggressive subtype with a MT rate exceeding 10% per year, characterized by heterogeneous or verrucous lesions involving multiple oral subsites⁴⁻⁶. To date, no therapies have impacted the natural history of this severe oral precancerous disease⁷⁻⁹, reflecting a critical unmet medical need.

Studies of the immune landscape led to pivotal trials of anti-PD-1 therapy in recurrent/metastatic head and neck squamous cell carcinoma (HNSCC)¹⁰⁻¹³. Applicant's prior retrospective study revealed a cytotoxic T-cell rich immune microenvironment in PVL¹⁴. These findings together with immunosurveillance studies in the context of lung premalignancy and of various immune-oncology (IO) interventions in preclinical models¹⁵⁻¹⁸ provided strong rationale for investigating PD-1/L1 axis blockade in oral precancerous disease. Applicant reports herein the first trial to evaluate the safety and efficacy of preventive anti-PD-1 therapy among patients with high-risk PVL.

This disclosure provides a method of treating a patient having a pre-cancerous oral leukoplakia (LK) lesion or delaying the progression of a pre-cancerous oral LK to oral squamous cell carcinoma, wherein the patient's sample comprising the LK lesion does not have 9p21.3 loss in the sample, the method comprising, or consisting of, or consisting essentially of administering an effective amount of an anti-PD-1 therapy to the patient. In one aspect, the oral LK comprises proliferative verrucous leukoplakia (PVL).

This disclosure provides a method of treating a patient having a pre-cancerous oral leukoplakia (LK) lesion, comprising, or consisting of, or consisting essentially of measuring for the presence of 9p21.3 loss in sample isolated from the patient (“patient sample”) comprising cells from the oral LK lesion isolated from the patient and administering effective amount of an anti-PD-1 treatment to the patient not a having 9p21.3 loss in their sample, or administering a therapy other than the anti-PD-1 treatment to a patient having 9p21.3 loss in their sample. In one aspect, the oral LK comprises proliferative verrucous leukoplakia (PVL).

Also provided is method of delaying the progression of pre-cancerous oral leukoplakia (LK) to oral squamous cell carcinoma in a patient having an oral LK lesion, the method comprising, or consisting essentially of, or yet further consisting of measuring 9p21.3 loss in patient sample comprising cells from the oral LK lesion isolated from the patient; and administering an effective amount of anti-PD-1 treatment to a patient not having 9p21.3 loss in the sample; or administering a therapy other than the anti-PD-1 treatment to a patient having 9p21.3 loss in the sample. In one aspect, the oral LK comprises proliferative verrucous leukoplakia (PVL).

Further provided is a method for predicting if a patient having a pre-cancerous oral leukoplakia (LK) lesion will respond to anti-PD-1 therapy, comprising measuring 9p21.3 loss in patient sample comprising cells from the oral LK lesion isolated from the patient, wherein a patient not having a 9p21.3 loss in the sample is likely to respond to anti-PD-1 therapy and a patient having a 9p21.3 loss in the sample is less likely to respond to anti-PD-1 therapy as compared to a patient having the 9p21.3 loss in the sample. In one aspect, the oral LK comprises proliferative verrucous leukoplakia (PVL).

### BRIEF DESCRIPTION OF THE DRAWINGS

FIGS. 1A-1C: Clinical and Pathologic Response. (FIG. 1A) Study flow diagram. (FIG. 1B) Waterfall plot showing best overall response rate to up to four doses of immunotherapy (nivolumab) in patients with high-risk oral proliferative verrucous leukoplakia (PVL). Response was determined by the relative change in composite score determined pre- to post-therapy using bidimensional lesion measurements and biopsy degree of dysplasia (mild, moderate, severe). A decrease by >80% equated to a major response (MR) a decrease by 40-80% (partial response, PR), and an increase by 10% or more or new carcinoma in situ (CIS) or oral squamous cell carcinoma (OSCC) development was deemed progressive disease (PD). Gray columns or bars indicate those patients with pre-treatment epithelial dysplasia tissue demonstrating a PD-L1 CPS (combined positive score) ≥20. Photographs of a responder (pre-treatment on the left, post-ICT on the right) showing a 4×3 cm verrucous lesion contiguous with a left maxillary gingival sulcus lesion defined by proliferative and hyperkeratotic change with inferior erythematous friability at a heaped-up border, which resolved post-immunotherapy. Of note, teeth #25-27 were extracted prior to the post-ICT photo being taken. (FIG. 1C) Swimmer plot showing key timepoints throughout the trial duration and follow-up period. Each row or bar represents an individual participant, followed left to right over time. All patients were alive at last follow-up.

FIGS. 2A-2B: Survival Outcomes. (FIG. 2A) Kaplan-Meier curve showing cancer-free survival (CFS) reported in months from the time of trial registration to the first of OSCC, death, or censored at last follow-up. (FIG. 2B) Forest plot showing the hazard ratio (HR) and 95% confidence intervals (log 10) of the impact of clinical and pathologic variables on CFS. Univariate Cox proportional hazard model. TMB=tumor mutational burden, M=male, F=female. HR>1: higher risk of CFS event.

FIGS. 3A-3B: Genomic Correlates of Response and Survival. (FIG. 3A) Mutational frequency plot with each column representing an individual dysplastic sample (sample IDs noted, CT #). Genes are arranged top to bottom organized by mutational frequency. Total mutational burden (TMB) for dysplastic samples is plotted in the bar graph above the mutational plot. The color key indicates the mutation type. Only the top 20 most frequently altered genes are displayed. (FIG. 3B) Copy-number alteration plot demonstrating allelic imbalance (amplifications, deletions), or both (polyploidy) among pre-treatment oral dysplastic epithelial samples prior to treatment with 4-doses of nivolumab arranged by those patients that developed oral carcinoma (left) vs. not (at last known follow-up). Amp=amplified or copy gain, del=deletion or copy loss.

FIG. 4: Measurement of Effect and Response Assessment.

FIGS. 5A-5C: Immunologic Correlates of Response and Survival. (FIG. 5A) Scatter dot and box plot showing PD-L1 combined positive score (CPS) values at baseline pre-treatment arranged by best overall response to nivolumab (using size and histologic composite scoring). MR=major response, PR=partial response, SD=stable disease, PD=progressive disease, UE=unevaluable. (FIG. 5B) Immunohistochemical staining for dysplastic tissue PD-L1 CPS (100× magnification shown). (FIG. 5C) Line plots depicting immune cell lineages in blood and in epithelial dysplastic tissue and T cell phenotypes in blood and in epithelial dysplastic tissue both pre-treatment (baseline) and on-treatment with nivolumab color-coded by response (using multiparametric immune profiling; flow cytometry). Percentage (%) values are expressed as a proportion of the total parent population of CD45+ viable cells within each blood or tissue sample. (*) adjusted p<0.001 (applying a Bonferroni-Dunn correction), Wilcoxon signed-rank test for paired samples (two-sided).

FIG. 6: is a table detailing clinical and pathologic assessments of all study patients. In the figure, SCC=squamous cell carcinoma, KUS=keratosis of undetermined significance without dysplasia, also referred to hyperkeratosis, not reactive; CR=complete response, PR=partial response, SD=stable disease, PD=progressive disease or carcinoma; (*) scores of 99 implies development of carcinoma at re-biopsy.

FIG. 7: is a table detailing adverse events potentially attributable to nivolumab (N=33). In the figure, A this row adds to N=32 because one patient had no post-baseline adverse events. Frequencies are shown above (%). Note: no grade 5 AEs were reported. Only grade 1-2 events reported in >10% of patients are listed, and all grade 3-4 events are listed. Abbreviations: AST=Aspartate aminotransferase, ALT=Alanine aminotransferase, GI=gastrointestinal.

### DETAILED DESCRIPTION

### Definitions

Throughout this disclosure, various publications, patents and published patent specifications are referenced by an identifying citation or by an Arabic numeral, the full citations for which are found immediately preceding the claims. The disclosures of these publications, patents and published patent specifications are hereby incorporated by reference into the present disclosure to more fully describe the state of the art to which this disclosure pertains.

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

All numerical designations, e.g., pH, temperature, time, concentration, and molecular weight, including ranges, are approximations which are varied (+) or (−) by increments of 1, 5, or 10%. It is to be understood, although not always explicitly stated that all numerical designations are preceded by the term “about.” It also is to be understood, although not always explicitly stated, that the reagents described herein are merely exemplary and that equivalents of such are known in the art.

As used herein, the term “comprising” is intended to mean that the methods include the recited elements, but not excluding others. “Consisting essentially of” when used to define methods, shall mean excluding other elements of any essential significance to the method. “Consisting of” shall mean excluding more than trace elements of other ingredients for claimed compositions and substantial method steps. Embodiments defined by each of these transition terms are within the scope of this disclosure. Accordingly, it is intended that the methods can include additional steps and components (comprising) or alternatively including steps of no significance (consisting essentially of) or alternatively, intending only the stated method steps (consisting of).

The term “subject,” “host,” “individual,” and “patient” are as used interchangeably herein to refer to animals, typically mammalian animals. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human. In some embodiments, a subject has or is diagnosed of having or is suspected of having a cancer.

As used herein, the term “sample isolated from a subject” or “test sample” refers to any liquid or solid material containing nucleic acids. In suitable embodiments, a test sample is obtained from a biological source (i.e., a “biological sample”), such as cells in culture or a tissue sample from an animal, preferably, a human. In some embodiments, a biological sample comprises a sample selected from blood, serum, plasma, a throat swab, a nasal swab, a nasopharyngeal wash, saliva, urine, gastric fluid, cerebrospinal fluid, tears, stool, mucus, sweat, earwax, oil, a glandular secretion, semen, vaginal fluid, interstitial fluids derived from tumorous tissue, ocular fluids, breath, hair, finger nails, skin, biopsy tissue, placental fluid, amniotic fluid, cord blood, lymphatic fluids, cavity fluids, sputum, pus, microbiota, meconium, breast milk, and other secretions or excretions. In a specific embodiment, the sample is a biopsy sample.

As used herein, “9p21.3” (3p14 or 17p13.1) loss means either focal or arm loss (i.e., deletion at 9p21.3 region could derive from arm or focal events), unless specifically qualified as “focal only.”

As used herein, the term “deletion or loss of a genomic region” as referred to in a context of 9p, any 9p cytoband (e.g. 9p21.3), as the presence of a genomic (DNA) copy number loss of the genomic region, using a cutoff between −0.15 and −0.3 for the log 2FC (log 2 fold change, where the fold change is the ratio between the copy number of the genomic region and the copy number of the rest of the genome).

The term “somatic copy-number alteration” (SCNA) intends an alteration in a gene copy number acquired by a cell that can be passed to the progeny of the mutated cell in the course of division. SCNA can be determined by methods known in the art. Non-limiting examples of such include fluorescent in situ hybridization, comparative genomic hybridization, array comparative genomic hybridization, single nucleotide polymorphism (SNP) array, genomic sequencing, high resolution microarray, and karyotype analysis. In one aspect, the SCNA is determined using a method comprising, consisting essentially of, or yet further consisting of SNP array

The term “determining” or “identifying” is to associate or affiliate a patient closely to a group or population of patients who likely experience the same or a similar clinical response to a therapy.

The term “selecting” a patient for a therapy refers to making an indication that the selected patient is suitable for the therapy. Such an indication can be made in writing by, for instance, a handwritten prescription or a computerized report making the corresponding prescription or recommendation.

“Having the same cancer” is used when comparing one patient to another or alternatively, one patient population to another patient population. For example, the two patients or patient population will each have or be suffering from a cancer for example, oral squamous cell carcinoma, head and neck cancer or colon cancer.

A “normal cell corresponding to the tumor tissue type” refers to a normal cell from a same tissue type as the tumor tissue. A non-limiting example is a normal lung cell from a patient having lung tumor, or a normal colon cell from a patient having colon tumor.

“Detecting, measuring or assessing” as used herein refers to determining the presence of a nucleic acid or gene of interest in a sample or the presence of a protein of interest in a sample. Detection does not require the method to provide 100% sensitivity and/or 100% specificity.

“Detectable label” as used herein refers to a molecule or a compound or a group of molecules or a group of compounds used to identify a nucleic acid or protein of interest. In some cases, the detectable label can be detected directly. In other cases, the detectable label can be a part of a binding pair, which can then be subsequently detected. Signals from the detectable label can be detected by various means and will depend on the nature of the detectable label. Detectable labels can be isotopes, fluorescent moieties, colored substances, and the like. Examples of means to detect detectable label include but are not limited to spectroscopic, photochemical, biochemical, immunochemical, electromagnetic, radiochemical, or chemical means, such as fluorescence, chemifluorescence, or chemiluminescence, or any other appropriate means.

The terms “oligonucleotide” or “polynucleotide” or “portion,” or “segment” thereof refer to a stretch of polynucleotide residues which is long enough to use in PCR or various hybridization procedures to identify or amplify identical or related parts of mRNA or DNA molecules. The polynucleotide compositions of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

When a genetic marker, is used as a basis for selecting a patient for a treatment described herein, the genetic marker is measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits; or (h) toxicity. As would be well understood by one in the art, measurement of the genetic marker in a clinical setting is a clear indication that this parameter was used as a basis for initiating, continuing, adjusting and/or ceasing administration of the treatments described herein.

In certain embodiments, the terms “disease” “disorder” and “condition” are used interchangeably herein, referring to a precancer or alternatively cancer, a status of being diagnosed with a cancer, or a status of being suspect of having a cancer. “Cancer”, which is also referred to herein as “tumor”, is a known medically as an uncontrolled division of abnormal cells in a part of the body, benign or malignant. Non-limiting examples of malignant neoplasms include a broad group of diseases involving unregulated cell division and growth, and invasion to nearby parts of the body. Non-limiting examples of cancers include carcinomas, sarcomas, leukemia, and lymphoma, e.g., colon cancer, colorectal cancer, rectal cancer, gastric cancer, melanoma, non-small cell lung cancer, small cell lung cancer, esophageal cancer, head and neck cancer, HPV negative head and neck cancer, breast cancer, brain cancer, lung cancer, stomach cancer, liver cancer, gall bladder cancer, or pancreatic cancer. In one embodiment, the term “cancer” refers to a solid tumor, which is an abnormal mass of tissue that usually does not contain cysts or liquid areas, including but not limited to, sarcomas, carcinomas, and certain lymphomas (such as Non-Hodgkin's lymphoma). In another embodiment, the term “cancer” refers to a liquid cancer, which is a cancer presenting in body fluids (such as, the blood and bone marrow), for example, leukemias (cancers of the blood) and certain lymphomas.

Additionally or alternatively, a cancer may refer to a local cancer (which is an invasive malignant cancer confined entirely to the organ or tissue where the cancer began), a metastatic cancer (referring to a cancer that spreads from its site of origin to another part of the body), a non-metastatic cancer, a primary cancer (a term used describing an initial cancer a subject experiences), a secondary cancer (referring to a metastasis from primary cancer or second cancer unrelated to the original cancer), an advanced cancer, an unresectable cancer, or a recurrent cancer.

Precancer cells or tumors are cells or tissue that contain abnormal cells that have an increased risk of turning cancerous.

Staging is the process of determining details about your cancer, such as tumor size and if it has spread. Typically, the stage guides treatment decisions. Stage I means the cancer is localized to the tissue where it originated. Stage II and III mean the cancer is larger and has grown into nearby tissues or lymph nodes. Stage IV indicates that the cancer cells are found in other organs from where the cancer originated.

In certain embodiments, the terms “disease” “disorder” and “condition” are used interchangeably herein, referring to a cancer, a status of being diagnosed with a cancer, or a status of being suspect of having a cancer. “Cancer”, which is also referred to herein as “tumor”, is a known medically as an uncontrolled division of abnormal cells in a part of the body, benign or malignant. In one embodiment, cancer refers to a malignant neoplasm, a broad group of diseases involving unregulated cell division and growth, and invasion to nearby parts of the body. Non-limiting examples of cancers include carcinomas, sarcomas, leukemia, and lymphoma, e.g., head and neck cancer, melanoma, colon cancer, colorectal cancer, rectal cancer, gastric cancer, esophageal cancer, head and neck cancer, breast cancer, brain cancer, lung cancer, stomach cancer, liver cancer, gall bladder cancer, or pancreatic cancer. In one embodiment, the term “cancer” refers to a solid tumor, which is an abnormal mass of tissue that usually does not contain cysts or liquid areas, including but not limited to, sarcomas, carcinomas, and certain lymphomas (such as oral squamous cell carcinoma, head and neck cancer or Non-Hodgkin's lymphoma). In another embodiment, the term “cancer” refers to a liquid cancer, which is a cancer presenting in body fluids (such as, the blood and bone marrow), for example, leukemias (cancers of the blood) and certain lymphomas.

Additionally or alternatively, a cancer may refer to a local cancer (which is an invasive malignant cancer confined entirely to the organ or tissue where the cancer began), a metastatic cancer (referring to a cancer that spreads from its site of origin to another part of the body), a non-metastatic cancer, a primary cancer (a term used describing an initial cancer a subject experiences), a secondary cancer (referring to a metastasis from primary cancer or second cancer unrelated to the original cancer), an advanced cancer, an unresectable cancer, or a recurrent cancer. As used herein, an advanced cancer refers to a cancer that had progressed after receiving one or more of: the first line therapy, the second line therapy, or the third line therapy.

Head and neck cancer (HNC) develops from tissues in the lip and oral cavity (mouth), the larynx (throat), salivary glands, nose, sinuses or the skin of the face. The most common types of head and neck cancers occur in the lip, mouth, and larynx. HNC may be associated with prior infection with high-risk types of HPV (e.g., HPV-16 and -18) and is responsible for HPV-positive HNC. HPV positive and negative tumors represent a different clinicopathological and molecular entities. Many HPV-negative HNC are tobacco and alcohol inducted and are characterized by TP53 mutation. See http://atlasgeneticsoncology.org/Tuimors/HeadNeckSCCID5078.html, last accessed on Jan. 29, 2022.

Oral squamous cell carcinoma refers to a cancer occurring between the vermillion border of the lips and he junction of the hard and soft palates or the posterior of one third of the tongue. See https://www.merckmanuals.com/professional/ear,-nose,-and-throat-disorders/tumors-of-the-head-and-neck/oral-squamous-cell-carcinoma, last accessed on Sep. 24, 2023.

The term “suitable for a therapy” or “suitably treated with a therapy” shall mean that the patient is likely to exhibit one or more desirable clinical outcomes as compared to patients having the same disease and receiving the same therapy but possessing a different characteristic that is under consideration for the purpose of the comparison. In one aspect, the characteristic under consideration is a genetic polymorphism or a somatic mutation. In another aspect, the characteristic under consideration is expression level of a gene or a polypeptide. In one aspect, a more desirable clinical outcome is relatively higher likelihood of or relatively better tumor response such as tumor load reduction. In another aspect, a more desirable clinical outcome is relatively longer overall survival. In yet another aspect, a more desirable clinical outcome is relatively longer progression free survival or time to tumor progression. In yet another aspect, a more desirable clinical outcome is relatively longer disease-free survival. In further another aspect, a more desirable clinical outcome is relative reduction or delay in tumor recurrence. In another aspect, a more desirable clinical outcome is relatively decreased metastasis. In another aspect, a more desirable clinical outcome is relatively lower relative risk. In yet another aspect, a more desirable clinical outcome is relatively reduced toxicity or side effects. In some embodiments, more than one clinical outcomes are considered simultaneously. In one such aspect, a patient possessing a characteristic, such as a genotype of a genetic polymorphism, can exhibit more than one more desirable clinical outcomes as compared to patients having the same disease and receiving the same therapy but not possessing the characteristic. As defined herein, the patient is considered suitable for the therapy. In another such aspect, a patient possessing a characteristic can exhibit one or more desirable clinical outcome but simultaneously exhibit one or more less desirable clinical outcome. The clinical outcomes will then be considered collectively, and a decision as to whether the patient is suitable for the therapy will be made accordingly, taking into account the patient's specific situation and the relevance of the clinical outcomes. In some embodiments, progression free survival or overall survival is weighted more heavily than tumor response in a collective decision making.

As used herein, the term “administration” and “administering” are used to mean introducing an agent into a subject. Routes of administration include, but are not limited to, oral (such as a tablet, capsule, or suspension), topical, transdermal, intranasal, vaginal, rectal, subcutaneous intravenous, intravenous, intraarterial, intramuscular, intraosseous, intraperitoneal, intraocular, subconjunctival, sub-Tenon's, intravitreal, retrobulbar, intracameral, intratumoral, epidural and intrathecal.

An “effective amount” is an amount sufficient to effect beneficial or desired results. An effective amount can be administered in one or more administrations, applications, or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. In general, one will desire to administer an amount of the compound that is effective to achieve a serum level commensurate with the concentrations found to be effective in vivo. These considerations, as well as effective formulations and administration procedures are well known in the art and are described in standard textbooks.

“Therapeutically effective amount” of a drug or an agent refers to an amount of the drug or the agent that is an amount sufficient to obtain a pharmacological response or alternatively, is an amount of the drug or agent that, when administered to a patient with a specified disorder or disease, is sufficient to have the intended effect, e.g., treatment, alleviation, amelioration, palliation, or elimination of one or more manifestations of the specified disorder or disease in the patient. A therapeutic effect does not necessarily occur by administration of one dose, and may occur only after administration of a series of doses. Thus, a therapeutically effective amount may be administered in one or more administrations.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of this technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, treatment excludes prophylaxis.

When the disease is cancer, the following clinical endpoints are non-limiting examples of treatment: (1) elimination of a cancer in a subject or in a tissue/organ of the subject or in a cancer loci; (2) reduction in tumor burden (such as number of cancer cells, number of cancer foci, number of cancer cells in a foci, size of a solid cancer, concentrate of a liquid cancer in the body fluid, and/or amount of cancer in the body); (3) stabilizing or delay or slowing or inhibition of cancer growth and/or development, including but not limited to, cancer cell growth and/or division, size growth of a solid tumor or a cancer loci, cancer progression, and/or metastasis (such as time to form a new metastasis, number of total metastases, size of a metastasis, as well as variety of the tissues/organs to house metastatic cells); (4) less risk of having a cancer growth and/or development; (5) inducing an immune response of the patient to the cancer, such as higher number of tumor-infiltrating immune cell, higher number of activated immune cells, or higher number cancer cell expressing an immunotherapy target, or higher level of expression of an immunotherapy target in a cancer cell; (6) higher probability of survival and/or increased duration of survival, such as increased overall survival (OS, which may be shown as 1-year, 2-year, 5-year, 10-year, or 20-year survival rate), increased progression free survival (PFS), increased disease free survival (DFS), increased time to tumor recurrence (TTR) and increased time to tumor progression (TTP). In some embodiments, the subject after treatment experiences one or more endpoints selected from tumor response, reduction in tumor size, reduction in tumor burden, increase in overall survival, increase in progression free survival, inhibiting metastasis, improvement of quality of life, minimization of drug-related toxicity, and avoidance of side-effects (e.g., decreased treatment emergent adverse events). In some embodiments, improvement of quality of life includes resolution or improvement of cancer-specific symptoms, such as but not limited to fatigue, pain, nausea/vomiting, lack of appetite, and constipation; improvement or maintenance of psychological well-being (e.g., degree of irritability, depression, memory loss, tension, and anxiety); improvement or maintenance of social well-being (e.g., decreased requirement for assistance with eating, dressing, or using the restroom; improvement or maintenance of ability to perform normal leisure activities, hobbies, or social activities; improvement or maintenance of relationships with family). In some embodiments, improved patient quality of life that is measured qualitatively through patient narratives or quantitatively using validated quality of life tools known to those skilled in the art, or a combination thereof. Additional non-limiting examples of endpoints include reduced hospital admissions, reduced drug use to treat side effects, longer periods off-treatment, and earlier return to work or caring responsibilities. In one aspect, prevention or prophylaxis is excluded from treatment.

Administration or treatment in “combination” refers to administering two agents such that their pharmacological effects are manifest at the same time. Combination does not require administration at the same time or substantially the same time, although combination can include such administrations.

The phrase “first line” or “second line” or “third line” etc., refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as primary therapy and primary treatment.” See National Cancer Institute website as www.cancer.gov, last visited on May 1, 2008. Typically, a patient is given a subsequent chemotherapy regimen because the patient did not shown a positive clinical or sub-clinical response to the first line therapy or the first line therapy has stopped.

The term “chemotherapy” or encompasses cancer therapies that employ chemical or biological agents or other therapies, such as radiation therapies, e.g., a small molecule drug or a large molecule, such as antibodies, Chimeric antigen receptor (CAR) therapies, RNAi and gene therapies. Non-limiting examples of chemotherapeutic agents are provided below. Unless specifically excluded, when a specific therapy is recited, equivalents of the therapy are within the scope of this disclosure.

An “immunotherapy agent” means a type of cancer treatment which uses a patient's own immune system to fight cancer, including but not limited to a physical intervene, a chemical substance, a biological molecule or particle, a cell, a tissue or organ, or any combinations thereof, enhancing or activating or initiating a patient's immune response against cancer. Non-limiting examples of immunotherapy agents include antibodies, immune regulators, checkpoint inhibitors, an antisense oligonucleotide (ASO), a RNA interference (RNAi), a Clustered Regularly Interspaced Short Palindromic Repeat (CRISPR) system, a viral vector, an anti-cancer cell therapy (e.g., transplanting an anti-cancer immune cell optionally amplified and/or activated in vivo, or administering an immune cell expressing a chimeric antigen receptor (CAR)), a CAR therapy, and cancer vaccines. As used herein, unless otherwise specified, an immunotherapy agent is not an inhibitor of thymidylate biosynthesis, or an anthracycline or other topoisomerase II inhibitor. As used herein, immune checkpoint refers to a regulator and/or modulator of the immune system (such as an immune response, an anti-tumor immune response, a nascent anti-tumor immune response, an anti-tumor immune cell response, an anti-tumor T cell response, and/or an antigen recognition of T cell receptor in the process of immune response). Their interaction activates either inhibitory or activating immune signaling pathways. Thus a checkpoint may contain one of the two signals: a stimulatory immune checkpoint that stimulates an immune response, and an inhibitory immune checkpoint inhibiting an immune response. In some embodiments, the immune checkpoint is crucial for self-tolerance, which prevents the immune system from attacking cells indiscriminately. However, some cancers can protect themselves from attack by stimulating immune checkpoint targets. In some embodiments, the immune checkpoints are present on T cells, antigen-presenting cells (APCs) and/or tumor cells.

One target of an immunotherapy agent is a tumor-specific antigen while the immunotherapy directs or enhances the immune system to recognize and attack tumor cells. Non-limiting examples of such agent includes a cancer vaccine presenting a tumor-specific antigen to the patient's immune system, a monoclonal antibody or an antibody-drug conjugate specifically binding to a tumor-specific antigen, a bispecific antibody specifically binding to a tumor-specific antigen and an immune cell (such as a T-cell engager or a NK-cell engager), an immune cell (such as a killer cell) specifically binding to a tumor-specific antigen (such as a CAR-T cell, a CAR-NK cell, and a CAR-NKT cell), a polynucleotide (or a vector comprising the same) transfecting/transducing an immune cell to express an tumor-specific antibody of an antigen binding fragment thereof (such as a CAR), or a polynucleotide (or a vector comprising the same) transfecting/transducing a cancer cell to express an antigen or a marker which can be recognized by an immune cell.

Another exemplified target is an inhibitory immune checkpoint which suppresses the nascent anti-tumor immune response, such as A2AR, B7-H3, B7-H4, BTLA, CTLA-4, CTLA-4/B7-1/B7-2, IDO, KIR, LAG3, NOX2, PD-1, PD-L1 and TIM-3, VISTA, SIGLEC7 (Sialic acid-binding immunoglobulin-type lectin 7, also designated as CD328) and SIGLEC9 (Sialic acid-binding immunoglobulin-type lectin 9, also designated as CD329). Non-limiting examples of such agent includes an antagonist or inhibitor of an inhibitory immune checkpoint, an agent reducing the expression and/or activity of an inhibitory immune checkpoint (such as via an antisense oligonucleotide (ASO), a RNA interference (RNAi), or a Clustered Regularly Interspaced Short Palindromic Repeat (CRISPR) system), an antibody or an antibody-drug conjugate or a ligand specifically binding to and reducing (or inhibiting) the activity of an inhibitory immune checkpoint, an immune cell with reduced (or inhibited) an inhibitory immune checkpoint (and optionally specifically binding to a tumor-specific antigen, such as a CAR-T cell, a CAR-NK cell, and a CAR-NKT cell), and a polynucleotide (or a vector comprising the same) transfecting/transducing an immune cell or a cancer cell to reduce or inhibit an inhibitory immune checkpoint thereof. Reducing expression or activity of such inhibitory immune checkpoint enhances immune response of a patient to a cancer.

An “anti-PD-1” therapy intends anti-PD-1 monoclonal antibodies that are a type of targeted immunotherapy called an immune checkpoint inhibitor. Checkpoint inhibitors don't kill cancer cells directly, instead, they target specific proteins on T-cells (a type of immune cell), one of which is called PD-1. Non-limiting examples of such are provided in the below Table 7 and Chen J, et al. (2020) Research Status and Outlook of PD-1/PD-L1 inhibitors for Cancer Therapy. Drug Des Devel Thor. 2020 Sep. 8; 14:3625-3649. doi: 10.2147/DDDT.S267433. PMID: 32982171; PMCID: PMC7490077. Without being bound by theory, Applicant intends that anti-PD-L1 antibodies would exhibit similar results as the ant-PD-1 antibody as disclosed herein.

TABLE 7

| Brand Name | Generic Name | Target |
|---|---|---|
| Opdivo (Bristol Myers Squibb) | Nivolumab | PD-1 |
| Keytruda (Merck) | Pembrolizumab | PD-1 |
| Libtayo (Regeneron) | Cemiplimab | PD-1 |
| Zynyz (Incyte) | Retifanlimab | PD-1 |
| Jemperli (GSK) | Dostarlimab | PD-1 |
| Imfinzi (AstraZeneca) | Durvalumab | CD274 (blocks |
| | | interaction of PD- |
| | | L1 with PD-1) |
| Bavencio (Pfizer) | Avelumab | PD-L1 (inhibits its |
| | | binding to PD-1) |
| Tecentriq (Genentech) | Atezolizumab | PD-L1 (inhibits its |
| | | binding to PD-1) |

Nivolumab is a therapeutic anti-PD-1 monoclonal antibody that acts as a checkpoint inhibitor and is sold under the brand name Opdivo™. It is also referred to as ONO-4538, BMS-936558 or MDX1106. As used herein, the term “Nivolumab” or “nivolumab” intends any antibody or fragment thereof that binds to the same epitope as Nivolumab or alternatively any antibody that comprises the same CDRs as Nivolumab, or competes with binding to it. Exemplary antibodies are disclosed in U.S. Pat. Nos. 8,008,449 and 8,779,15.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits, rat, canine, donkey, mice, camelids (such as dromedaries, llamas, and alpacas), as well as non-mammalian species, such as shark immunoglobulins. Unless specifically noted otherwise, the term “antibody” includes intact immunoglobulins and “antibody fragments” or “antigen binding fragments” that specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴M⁻¹ greater or at least 10⁵ M⁻¹ greater than a binding constant for other molecules in a biological sample). The term “antibody” also includes genetically engineered forms such as chimeric antibodies (for example, murine or humanized non-primate antibodies), heteroconjugate antibodies (such as, bispecific antibodies). See also, Pierce Catalog and Handbook, 1994-1995 (Pierce Chemical Co., Rockford, Ill.); Owen et al., Kuby Immunology, 7^{th} Ed., W.H. Freeman & Co., 2013; Murphy, Janeway's Immunobiology, 8^{th} Ed., Garland Science, 2014; Male et al., Immunology (Roitt), 8^{th} Ed., Saunders, 2012; Parham, The Immune System, 4^{th}, Ed., Garland Science, 2014. The term “antibody” includes any protein or peptide containing molecule that comprises at least a portion of an immunoglobulin molecule, such as the whole antibody and any antigen binding fragment or a single chain thereof. The terms “antibody,” “antibodies” and “immunoglobulin” also include immunoglobulins of any isotype, fragments of antibodies which retain specific binding to antigen, including, but not limited to, Fab, Fab′, F(ab)₂, Fv, scFv, dsFv, Fd fragments, dAb, VH, VL, VhH, and V-NAR domains; minibodies, diabodies, triabodies, tetrabodies and kappa bodies; multispecific antibody fragments formed from antibody fragments and one or more isolated. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, at least one portion of a binding protein, chimeric antibodies, humanized antibodies, single-chain antibodies, and fusion proteins comprising an antigen-binding portion of an antibody and a non-antibody protein. The variable regions of the heavy and light chains of the immunoglobulin molecule contain a binding domain that interacts with an antigen. The constant regions of the antibodies (Abs) may mediate the binding of the immunoglobulin to host tissues. The antibodies can be polyclonal, monoclonal, multispecific (e.g., bispecific antibodies), and antibody fragments, so long as they exhibit the desired biological activity.

As used herein, the term “monoclonal antibody” refers to an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

In some embodiments, the antibody is a bispecific immune cell engager, referring to a bispecific monoclonal antibody that is capable of recognizing and specifically binding to a tumor antigen (such as CD19, EpCAM, MCSP, HER2, EGFR or CS-1) and an immune cell, and directing an immune cell to cancer cells, thereby treating a cancer. Non-limiting examples of such antibody include bispecific T cell engager, bispecific cytotoxic T lymphocytes (CTL) engager, and bispecific NK cell engager. In one embodiment, the engager is a fusion protein consisting of two single-chain variable fragments (scFvs) of different antibodies. Additionally, or alternatively, the immune cell is a killer cell, including but not limited to: a cytotoxic T cell, a gamma delta T cell, a NK cell and a NK-T cell.

The term “chimeric antigen receptor” (CAR), as used herein, refers to a fused protein comprising an extracellular domain capable of binding to an antigen, a transmembrane domain derived from a polypeptide different from a polypeptide from which the extracellular domain is derived, and at least one intracellular domain. The “chimeric antigen receptor (CAR)” is sometimes called a “chimeric receptor”, a “T-body”, or a “chimeric immune receptor (CIR).” The “extracellular domain capable of binding to an antigen” means any oligopeptide or polypeptide that can bind to a certain antigen. The “intracellular domain” or “intracellular signaling domain” means any oligopeptide or polypeptide known to function as a domain that transmits a signal to cause activation or inhibition of a biological process in a cell. In certain embodiments, the intracellular domain may comprise, alternatively consist essentially of, or yet further comprise one or more costimulatory signaling domains in addition to the primary signaling domain. The “transmembrane domain” means any oligopeptide or polypeptide known to span the cell membrane and that can function to link the extracellular and signaling domains. A chimeric antigen receptor may optionally comprise a “hinge domain” which serves as a linker between the extracellular and transmembrane domains.

As used herein, the term “T cell,” refers to a type of lymphocyte that matures in the thymus. T cells play an important role in cell-mediated immunity and are distinguished from other lymphocytes, such as B cells, by the presence of a T-cell receptor on the cell surface. T-cells for using in a cell therapy and/or a CAR therapy may either be isolated or obtained from a commercially available source. “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses.

As used herein, the term “NK cell,” also known as natural killer cell, refers to a type of lymphocyte that originates in the bone marrow and play a critical role in the innate immune system. NK cells provide rapid immune responses against viral-infected cells, tumor cells or other stressed cell, even in the absence of antibodies and major histocompatibility complex on the cell surfaces. NK cells for using in a cell therapy and/or a CAR therapy may either be isolated or obtained from a commercially available source.

The term “clinical outcome”, “clinical parameter”, “clinical response”, or “clinical endpoint” refers to any clinical observation or measurement relating to a patient's reaction to a therapy. Non-limiting examples of clinical outcomes include tumor response (TR), overall survival (OS), progression free survival (PFS), disease free survival, time to tumor recurrence (TTR), time to tumor progression (TTP), relative risk (RR), toxicity or side effect.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at cancer.gov. Typically, a patient is given a subsequent therapy because the patient did not show a positive clinical or sub-clinical response to the first line therapy or the first line therapy has stopped.

The term “adjuvant” therapy refers to administration of a therapy or chemotherapeutic regimen to a patient in addition to the primary or initial treatment, such as after removal of a tumor by surgery. Adjuvant therapy is typically given to minimize or prevent a possible cancer reoccurrence. Alternatively, “neoadjuvant” therapy refers to administration of therapy or chemotherapeutic regimen before surgery, typically in an attempt to shrink the tumor prior to a surgical procedure to minimize the extent of tissue removed during the procedure. Additionally, or alternatively, such adjuvant therapy potentials (i.e., sensitizes the subject to the original therapy) the subject may help reach one or more of clinical endpoints of the cancer treatment.

An “immunotherapy agent” means a type of cancer treatment which uses a patient's own immune system to fight cancer, including but not limited to a physical intervene, a chemical substance, a biological molecule or particle, a cell, a tissue or organ, or any combinations thereof, enhancing or activating or initiating a patient's immune response against cancer. Non-limiting examples of immunotherapy agents include antibodies, immune regulators, checkpoint inhibitors, an antisense oligonucleotide (ASO), a RNA interference (RNAi), a Clustered Regularly Interspaced Short Palindromic Repeat (CRISPR) system, a viral vector, an anti-cancer cell therapy (e.g., transplanting an anti-cancer immune cell optionally amplified and/or activated in vivo, or administering an immune cell expressing a chimeric antigen receptor (CAR)), a CAR therapy, and cancer vaccines. As used herein, unless otherwise specified, an immunotherapy agent is not an inhibitor of thymidylate biosynthesis, or an anthracycline or other topoisomerase II inhibitor. As used herein, immune checkpoint refers to a regulator and/or modulator of the immune system (such as an immune response, an anti-tumor immune response, a nascent anti-tumor immune response, an anti-tumor immune cell response, an anti-tumor T cell response, and/or an antigen recognition of T cell receptor in the process of immune response). Their interaction activates either inhibitory or activating immune signaling pathways. Thus, a checkpoint may contain one of the two signals: a stimulatory immune checkpoint that stimulates an immune response, and an inhibitory immune checkpoint inhibiting an immune response. In some embodiments, the immune checkpoint is crucial for self-tolerance, which prevents the immune system from attacking cells indiscriminately. However, some cancers can protect themselves from attack by stimulating immune checkpoint targets. In some embodiments, the immune checkpoints are present on T cells, antigen-presenting cells (APCs) and/or tumor cells.

One target of an immunotherapy agent is a tumor-specific antigen while the immunotherapy directs or enhances the immune system to recognize and attack tumor cells. Non-limiting examples of such agent includes a cancer vaccine presenting a tumor-specific antigen to the patient's immune system, a monoclonal antibody or an antibody-drug conjugate specifically binding to a tumor-specific antigen, a bispecific antibody specifically binding to a tumor-specific antigen and an immune cell (such as a T-cell engager or a NK-cell engager), an immune cell (such as a killer cell) specifically binding to a tumor-specific antigen (such as a CAR-T cell, a CAR-NK cell, and a CAR-NKT cell), a polynucleotide (or a vector comprising the same) transfecting/transducing an immune cell to express an tumor-specific antibody of an antigen binding fragment thereof (such as a CAR), or a polynucleotide (or a vector comprising the same) transfecting/transducing a cancer cell to express an antigen or a marker which can be recognized by an immune cell.

Another exemplified target is an inhibitory immune checkpoint which suppresses the nascent anti-tumor immune response, such as A2AR, B7-H3, B7-H4, BTLA, CTLA-4, CTLA-4/B7-1/B7-2, IDO, KIR, LAG3, NOX2, PD-1, PD-L1 and TIM-3, VISTA, SIGLEC7 (Sialic acid-binding immunoglobulin-type lectin 7, also designated as CD328) and SIGLEC9 (Sialic acid-binding immunoglobulin-type lectin 9, also designated as CD329). Non-limiting examples of such agent includes an antagonist or inhibitor of an inhibitory immune checkpoint, an agent reducing the expression and/or activity of an inhibitory immune checkpoint (such as via an antisense oligonucleotide (ASO), a RNA interference (RNAi), or a Clustered Regularly Interspaced Short Palindromic Repeat (CRISPR) system), an antibody or an antibody-drug conjugate or a ligand specifically binding to and reducing (or inhibiting) the activity of an inhibitory immune checkpoint, an immune cell with reduced (or inhibited) an inhibitory immune checkpoint (and optionally specifically binding to a tumor-specific antigen, such as a CAR-T cell, a CAR-NK cell, and a CAR-NKT cell), and a polynucleotide (or a vector comprising the same) transfecting/transducing an immune cell or a cancer cell to reduce or inhibit an inhibitory immune checkpoint thereof. Reducing expression or activity of such inhibitory immune checkpoint enhances immune response of a patient to a cancer.

A further possible immunotherapy target is a stimulatory checkpoint molecule (including but not limited to 4-1BB, CD27, CD28, CD40, CD122, CD137, OX40, GITR and ICOS), wherein the immunotherapy agent actives or enhances the anti-tumor immune response. Non-limiting examples of such agent includes an agonist of a stimulatory checkpoint, an agent increasing the expression and/or activity of a stimulating immune checkpoint, an antibody or an antibody-drug conjugate or a ligand specifically binding to and activating or enhancing the activity of a stimulating immune checkpoint, an immune cell with increased expression and/or activity of a stimulating immune checkpoint (and optionally specifically binding to a tumor-specific antigen, such as a CAR-T cell, a CAR-NK cell, and a CAR-NKT cell), and a polynucleotide (or a vector comprising the same) transfecting/transducing an immune cell or a cancer cell to express a stimulating immune checkpoint thereof.

As used herein the term “PD-1” refers to a specific protein fragment associated with this name and any other molecules that have analogous biological function that share at least 70%, or alternatively at least 80% amino acid sequence identity, or alternatively 90% sequence identity, or alternatively at least 95% sequence identity with the PD-1 sequence as shown herein and/or a suitable binding partner of PD-L1. Non-limiting example sequences of PD-1 are provided herein, such as but not limited to those under the following reference numbers—GCID:GC02M241849; HGNC: 8760; Entrez Gene: 5133; Ensembl: ENSG00000188389; OMIM: 600244; and UniProtKB: Q15116—and the sequence: MQIPQAPWPVVWAVLQLGWRPGWFLDSPDRPWNPPTFSPALLVVTEGDNATFTCSF SNTSESFVLNWYRMSPSNQTDKLAAFPEDRSQPGQDCRFRVTQLPNGRDFHMSVVR ARRNDSGTYLCGAISLAPKAQIKESLRAELRVTERRAEVPTAHPSPSPRPAGQFQTLV VGVVGGLLGSLVLLVWVLAVICSRAARGTIGARRTGQPLKEDPSAVPVFSVDYGEL DFQWREKTPEPPVPCVPEQTEYATIVFPSGMGTSSPARRGSADGPRSAQPLRPEDGHC SWPL, and equivalents thereof. Non-limiting examples of commercially available antibodies thereto include pembrolizumab (Merck), nivolumab (Bristol-Myers Squibb), pidilizumab (Cure Tech), AMP-224 (GSK), AMP-514 (GSK), PDR001 (Novartis), and cemiplimab (Regeneron and Sanofi).

As used herein the term “PD-L1” refers to a specific protein fragment associated with this name and any other molecules that have analogous biological function that share at least 70%, or alternatively at least 80% amino acid sequence identity, or alternatively 90% sequence identity, or alternatively at least 95% sequence identity with the PD-L1 sequence as shown herein and/or an suitable binding partner of PD-1. Non-limiting example sequences of PD-L1 are provided herein, such as but not limited to those under the following reference numbers—GCID: GC09P005450; HGNC: 17635; Entrez Gene: 29126; Ensembl: ENSG00000120217; OMIM: 605402; and UniProtKB: Q9NZQ7—and the sequence: MRIFAVFIFMTYWHLLNAFTVTVPKDLYVVEYGSNMTIECKFPVEKQLDLAALIVY WEMEDKNIIQFVHGEEDLKVQHSSYRQRARLLKDQLSLGNAALQITDVKLQDAGVY RCMISYGGADYKRITVKVNAPYNKINQRILVVDPVTSEHELTCQAEGYPKAEVIWTS SDHQVLSGKTTTTNSKREEKLFNVTSTLRINTTTNEIFYCTFRRLDPEENHTAELVIPE LPLAHPPNERTHLVILGAILLCLGVALTFIFRLRKGRMMDVKKCGIQDTNSKKQSDT HLEET, and equivalents thereof. Non-limiting examples of commercially available antibodies thereto include atezolizumab (Roche Genentech), avelumab (Merck Soreno and Pfizer), durvalumab (AstraZeneca), BMS-936559 (Bristol-Myers Suibb), and CK-301 (Checkpoint Therapeutics).

Additional or alternative targets may be utilized by an immunotherapy agent, such as an immune regulating agent, including but not limited to, an agent activating an immune cell, an agent recruiting an immune cell to a cancer or a cancer cell, or an agent increasing immune cell infiltrated into a solid tumor and/or a cancer loci. Non-limiting examples of such agent is an immune regulator or a variant, a mutant, a fragment, an equivalent thereof.

In some embodiments, an immunotherapy agent utilizes one or more targets, such as a bispecific T cell engager, a bispecific NK cell engager, or a CAR cell therapy. In some embodiments, the immunotherapy agent targets one or more immune regulatory or effector cells.

A “tumor response” (TR) refers to a tumor's response to therapy. A “complete response” (CR) to a therapy refers to the clinical status of a patient with evaluable but non-measurable disease, whose tumor and all evidence of disease have disappeared following administration of the therapy. In this context, a “partial response” (PR) refers to a response that is anything less than a complete response. “Stable disease” (SD) indicates that the patient is stable following the therapy. “Progressive disease” (PD) indicates that the tumor has grown (i.e., become larger) or spread (i.e., metastasized to another tissue or organ) or the overall cancer has gotten worse following the therapy. For example, tumor growth of more than 20 percent since the start of therapy typically indicates progressive disease. “Non-response” (NR) to a therapy refers to status of a patient whose tumor or evidence of disease has remained constant or has progressed.

“Overall Survival” (OS) refers to the length of time of a cancer patient remaining alive following a cancer therapy.

“Progression free survival” (PFS) or “Time to Tumor Progression” (TTP) refers to the length of time following a therapy, during which the tumor in a cancer patient does not grow.

Progression-free survival includes the amount of time a patient has experienced a complete response, partial response, or stable disease.

“Disease free survival” refers to the length of time following a therapy, during which a cancer patient survives with no signs of the cancer or tumor.

“Time to Tumor Recurrence (TTR)” refers to the length of time, following a cancer therapy such as surgical resection or chemotherapy, until the tumor has reappeared (come back). The tumor may come back to the same place as the original (primary) tumor or to another place in the body.

“Relative Risk” (RR), in statistics and mathematical epidemiology, refers to the risk of an event (or of developing a disease) relative to exposure. Relative risk is a ratio of the probability of the event occurring in the exposed group versus a non-exposed group.

One chemotherapy is 5-Fluorouracil (5-FU) which belongs to the family of therapy drugs called pyrimidine based anti-metabolites. It is a pyrimidine analog, which is transformed into different cytotoxic metabolites that are then incorporated into DNA and RNA thereby inducing cell cycle arrest and apoptosis. Chemical equivalents are pyrimidine analogs which result in disruption of DNA replication. Chemical equivalents inhibit cell cycle progression at S phase resulting in the disruption of cell cycle and consequently apoptosis. Equivalents to 5-FU include prodrugs, analogs and derivative thereof such as 5′-deoxy-5-fluorouridine (doxifluoroidine), 1-tetrahydrofuranyl-5-fluorouracil (ftorafur), capecitabine (Xeloda®), S-1 (MBMS-247616, consisting of tegafur and two modulators, a 5-chloro-2,4-dihydroxypyridine and potassium oxonate), ralititrexed (tomudex), nolatrexed (Thymitaq, AG337), LY231514 and ZD9331, as described for example in Papamichael (1999) The Oncologist 4:478-487.

Cetuximab or Erbitux (commercially available from Lily) is an FDA-approved antibody to the epidermal growth factor receptor (EGFR) that is used alone or in combination with irinotecan (also known as CPT-11 or Camptosar) to treat various cancers. See https://chemocare.com/chemotherapy/drug-info/cetuximab.aspx.

Another chemotherapy is 5-FU based adjuvant therapy which refers to 5-FU alone or alternatively the combination of 5-FU with one or more other treatments, that include, but are not limited to radiation, methyl-CCNU, leucovorin, oxaliplatin (such as cisplatin), irinotecan, mitomycin, cytarabine, doxorubicin, cyclophosphamide, and levamisole, as well as an immunotherapy. Specific treatment adjuvant regimens are known in the art such as weekly Fluorouracil/Leucovorin, weekly Fluorouracil/Leucovorin+Bevacizumab, FOLFOX, FOLFOX-4, FOLFOX6, modified FOLFOX6 (mFOLFOX6), FOLFOX6 with bevacizumab, mFOLFOX6+Cetuximab, mFOLFOX6+Panitumumab, modified FOLFOX7 (mFOLFOX7), FOLFIRI, FOLFIRI with Bevacizumab, FOLFIRI+Ziv-aflibercept, FOLFIRI with Cetuximab, FOLFIRI+Panitumumab, FOLFIRI+Ramucirumab, FOLFOXIRI, FOLFIRI with FOLFOX6, FOLFOXIRI+Bevacizumab, FOLFOXIRI+Cetuximab, FOLFOXIRI+Panitumumab, Roswell Park Fluorouracil/Leucovorin, Roswell Park Fluorouracil/Leucovorin+Bevacizumab, Simplified Biweekly Infusional Fluorouracil/Leucovorin, Simplified Biweekly Infusional Fluorouracil/Leucovorin+Bevacizumab, and MOF (semustine (methyl-CCNU), vincrisine (Oncovin®) and 5-FU). For a review of these therapies see Beaven and Goldberg (2006) Oncology 20(5):461-470 as well as www.cancertherapyadvisor.com/home/cancer-topics/gastrointestinal-cancers/gastrointestinal-cancers-treatment-regimens/colon-cancer-treatment-regimens/. Other chemotherapeutics can be added, e.g., oxaliplatin or irinotecan.

Capecitabine is chemotherapy that is a prodrug of (5-FU) that is converted to its active form by the tumor-specific enzyme PynPase following a pathway of three enzymatic steps and two intermediary metabolites, 5′-deoxy-5-fluorocytidine (5′-DFCR) and 5′-deoxy-5-fluorouridine (5′-DFUR). Capecitabine is marketed by Roche under the trade name Xeloda®.

Leucovorin (Folinic acid) is a chemotherapy which is an adjuvant used in cancer therapy. It is used in synergistic combination with 5-FU to improve efficacy of the chemotherapeutic agent. Without being bound by theory, addition of Leucovorin is believed to enhance efficacy of 5-FU by inhibiting thymidylate synthase. It has been used as an antidote to protect normal cells from high doses of the anticancer drug methotrexate and to increase the antitumor effects of fluorouracil (5-FU) and tegafur-uracil. It is also known as citrovorum factor and Wellcovorin. This compound has the chemical designation of L-Glutamic acid N-[4-[[(2-amino-5-formyl-1,4,5,6,7,8-hexahydro-4-oxo-6-pteridinyl)methyl]amino]benzoyl], calcium salt (1:1).

“Oxaliplatin” (Eloxatin) is a chemotherapy that is a platinum-based chemotherapy drug in the same family as cisplatin and carboplatin. It is typically administered in combination with fluorouracil and leucovorin in a combination known as FOLFOX for the treatment of colorectal cancer. Compared to cisplatin, the two amine groups are replaced by cyclohexyldiamine for improved antitumor activity. The chlorine ligands are replaced by the oxalato bidentate derived from oxalic acid in order to improve water solubility. Equivalents to Oxaliplatin are known in the art and include, but are not limited to cisplatin, carboplatin, aroplatin, lobaplatin, nedaplatin, and JM-216 (see McKeage et al. (1997) J. Clin. Oncol. 201:1232-1237 and in general, Chemotherapy for Gynecological Neoplasm, Curr. Therapy and Novel Approaches, in the Series Basic and Clinical Oncology, Angioli et al. Eds., 2004).

“FOLFOX” is chemotherapy that is an abbreviation for a type of combination therapy that is used to treat cancer. This therapy includes leucovorin (“FOL”), 5-FU (“F”), and oxaliplatin (“OX”) and encompasses various regimens, such as FOLFOX-4, FOLFOX-6, modified FOLOX-6, and FOLFOX-7, which vary in doses and ways in which each of the three drugs are administered. “FOLFIRI” is an abbreviation for a type of combination therapy that is used treat cancer and comprises, or alternatively consists essentially of, or yet further consists of 5-FU, leucovorin, and irinotecan. Information regarding these treatments are available on the National Cancer Institute's web site, cancer.gov, last accessed on May 30, 2020 as well as www.cancertherapyadvisor.com/home/cancer-topics/gastrointestinal-cancers/gastrointestinal-cancers-treatment-regimens/colon-cancer-treatment-regimens/, last accessed on May 30, 2020.

Irinotecan (CPT-11) is a chemotherapy sold under the trade name of Camptosar. It is a semi-synthetic analogue of the alkaloid camptothecin, which is activated by hydrolysis to SN-38 and targets topoisomerase I. Chemical equivalents are those that inhibit the interaction of topoisomerase I and DNA to form a catalytically active topoisomerase I-DNA complex. Chemical equivalents inhibit cell cycle progression at G2-M phase resulting in the disruption of cell proliferation.

S-1 is a chemotherapy that consists of three agents (at a molar ratio of 1:0.4:1): tegafur, 5-chloro-2-4-dihydroxypyridine, and potassium oxonate.

An “antifolate” is a drug or biologic chemotherapy that impairs the function of folic acids, e.g., an antimetabolite agent that inhibits the use of a metabolite, i.e., another chemical that is part of normal metabolism. In cancer treatment, antimetabolites interfere with DNA production, thus cell division and growth of the tumor. Non-limiting examples of these agents are dihydrofolate reductase inhibitors, such as methotrexate, Aminopterin, and Pemetrexed; thymidylate synthase inhibitors, such as Raltitrexed or Pemetrexed; purine based, i.e. an adenosine deaminase inhibitor, such as Pentostatin, a thiopurine, such as Thioguanine and Mercaptopurine, a halogenated/ribonucleotide reductase inhibitor, such as Cladribine, Clofarabine, Fludarabine, or a guanine/guanosine: thiopurine, such as Thioguanine; or Pyrimidine based, i.e. cytosine/cytidine: hypomethylating agent, such as Azacitidine and Decitabine, a DNA polymerase inhibitor, such as Cytarabine, a ribonucleotide reductase inhibitor, such as Gemcitabine, or a thymine/thymidine: thymidylate synthase inhibitor, such as a Fluorouracil (5-FU).

### DESCRIPTIVE EMBODIMENTS

PL demonstrates a rich immune microenvironment, providing strong rationale to evaluate anti-PD-1 immune-checkpoint therapy. The first clinical trial reported [ESMO; August 2022 by Glenn Hanna] of an ICT in PL was a single-arm, phase 2 trial investigated nivolumab (N) among patients (pts) with PL (multifocal, contiguous, or a single lesion ≥4 cm with any degree of epithelial dysplasia). As disclosed herein, patients underwent pre-treatment biopsy of 1-3 sites then received 4 doses of nivolumab (“N”) (480 mg IV) every 28-days, followed by re-biopsy. Intraoral photographs and bidirectional measurements occurred at each visit. Primary endpoint was a change in composite score (size and degree of dysplasia) pre- to post-treatment (complete response: >80% decrease in score; partial response: 40-80% decrease). Secondary endpoints: safety, cancer-free survival (CFS). Median DFI for those with a prior head and neck cancer diagnosis: 10.5 months (0.3-195). Time from study registration to first dose of nivolumab: 9 days (0-42). Median # of nivolumab cycles delivered: 4 (12% received <4 doses). Median time from first dose of nivolumab to post-treatment biopsy (end of treatment): 115 days (29-171).

To the best of Applicant's knowledge, this is the first study to demonstrate the efficacy of anti-PD-1 blockade among patients with high-risk oral leukoplakia/PL. There was an acceptable toxicity profile in a non-cancer population. There was evidence of disease regression among more than one-third of participants. Clinical benefit was observed regardless of PD-L1 status. Applicant also observed favorable 2-year CFS of 78% among this high-risk population. Thus, in addition to identifying high risk individuals, Applicant provides a treatment comprising an immunotherapy as a preventive strategy for selected patients.

Genomic somatic copy number alterations (SCNAs), including copy-number losses of on chromosome 9p21.3 were recently shown to drive oral precancer invasive-disease transition to oral squamous-cell cancer (SCC), correlate with immune-cold tumor microenvironments (TMEs) and/or ICI resistance HPV⁻ subtype of head and neck squamous-cell carcinoma (HNSC), including oral SCC, the most common site of HPV⁻ subtype of head and neck squamous-cell carcinoma (HNSC).

Applicant has demonstrated that oral precancers harboring 9p21 deletion exhibit the highest cancer risk and are associated with an immune-hot phenotype that undergoes an aneuploid switch to immune-depleted invasive cancers during malignant transformation [MT]. Without being bound by theory, this phenomenon is hypothesized to be mediated in part by an increase in size of chromosome 9p21 deletion to include other loci (such as the CD274/PD-L1 gene locus 9p24), and/or epistatic interactions with 17p13 loss/TP53 mutations. In this nivolumab-treated clinical protocol cohort, all (6/6) patients who developed cancer had 9p21 loss at baseline, and it is possible that resistance to the PD-1 inhibitor could have arisen during treatment as a result of 9p deletion size expansion to include 9p24, leading to downregulation of expression of the therapeutic target (i.e., PD-L1), and immune depletion.

Perhaps the most notable finding was the presence of 9p21.3 chromosomal loss in all DNA whole-exome sequenced pre-treatment oral PL specimens among patients that later developed oral cancer post-nivolumab treatment. 9p21 LOH has been reported in 45% of PL patients. The PD-L1 gene (CD274) exists on chromosome 9p, and it has been shown that 9p arm-level loss may predict poor PD-1 inhibitor oral cancer response and trigger interactions to overcome a proimmunogenic checkpoint signal. Together these data support a mechanism of anti-PD-1 resistance with the presence of 9p21 LOH.

Applicant's disclosure has enabled the identification of the specific genetic region (at the level of chromosome 9p21.3) which determine PL resistance to ICT immunotherapy, and potentially the first predictive genomic marker for precision therapy in any precancer/prevention/interception setting. Furthermore, this discovery has improved the biologic understanding of how immunotherapy works, which is vital for the design and development of new improved immunotherapeutic drugs.

Applicant's disclosure and methods can spare patients from debilitating oral neoplastic disease and morbidity of oral tumor resections on vital head and neck structures like the tongue, swallowing function, cosmetic deforming disease and its treatment, quality of life and overall survival by qualifying those that may respond to immunotherapy.

This disclosure also addresses a major physician need when considering immunotherapy treatment for patients diagnosed with squamous precancers by predicting patient response to anti PD-1 ICTs, the major class of immunotherapy for HNSC and all other solid tumors with FDA-approved ICIs.

Applicant's disclosure led to the finding of a link between 9p21 loss and ICT resistance. These findings also identified a possible epistatic interaction between 9p21 loss and 17p13 loss based on study of early stage oral squamous cell carcinoma (SCC), in the current PL data Applicant found that the extremes of cancer development from progression and cancer development in <2 months had concurrent 17p13 loss, vs the patient with cancer development >24 months later, had isolated 9p21 loss alone.

Although PD-1 ICTs represent a major breakthrough in cancer treatment, only ˜15% of patients HPV-negative HNSC (and other squamous-tumors) patients respond durably to this class of therapies and there are very limited and suboptimal predictors of response/resistance for ICTs. Applicant provides the first reported ICT trial in any precancer, so 9p21 is the first/sole predictive biomarker for ICT in precancer. This represents a tremendous unmet clinical/medical need for the most widely used class of cancer therapy drugs now and for the forseeable future. Therefore, the ability to predict the patient's response (using 9p alteration patterns) to this class of therapies is a unique and novel discovery. Knowing who will and won't respond to ICTs avoids losing precious months of limited survival time in recurrent HNSC to useless/ineffective therapy at huge financial costs, and quality/quantity of life.

### Diagnostic, Prognostic and Therapeutic Methods

The disclosure further provides diagnostic, prognostic, and therapeutic methods, which are based, at least in part, on determination of the identity of a genotype of interest identified herein.

For example, information obtained using the diagnostic assays described herein is useful for determining if a subject is suitable for cancer treatment of a given type. Based on the prognostic information, a doctor can recommend a therapeutic protocol, useful for reducing the malignant mass or tumor in the patient or treat cancer in the individual.

A patient's likely clinical outcome following a clinical procedure such as a therapy or surgery can be expressed in relative terms. For example, a patient having a particular genotype or expression level can experience relatively longer overall survival than a patient or patients not having the genotype or expression level. The patient having the particular genotype or expression level, alternatively, can be considered as likely to survive. Similarly, a patient having a particular genotype or expression level can experience relatively longer progression free survival, or time to tumor progression, than a patient or patients not having the genotype or expression level. The patient having the particular genotype or expression level, alternatively, can be considered as not likely to suffer tumor progression. Further, a patient having a particular genotype or expression level can experience relatively shorter time to tumor recurrence than a patient or patients not having the genotype or expression level. The patient having the particular genotype or expression level, alternatively, can be considered as not likely to suffer tumor recurrence. Yet in another example, a patient having a particular genotype or expression level can experience relatively more complete response or partial response than a patient or patients not having the genotype or expression level. The patient having the particular genotype or expression level, alternatively, can be considered as likely to respond. Accordingly, a patient that is likely to survive, or not likely to suffer tumor progression, or not likely to suffer tumor recurrence, or likely to respond following a clinical procedure is considered suitable for the clinical procedure.

It is to be understood that information obtained using the diagnostic assays described herein can be used alone or in combination with other information, such as, but not limited to, genotypes or expression levels of other genes, clinical chemical parameters, histopathological parameters, or age, gender, and weight of the subject. When used alone, the information obtained using the diagnostic assays described herein is useful in determining or identifying the clinical outcome of a treatment, selecting a patient for a treatment, or treating a patient, etc. When used in combination with other information, on the other hand, the information obtained using the diagnostic assays described herein is useful in aiding in the determination or identification of clinical outcome of a treatment, aiding in the selection of a patient for a treatment, or aiding in the treatment of a patient and etc. In a particular aspect, the genotypes or expression levels of one or more genes as disclosed herein are used in a panel of genes, each of which contributes to the final diagnosis, prognosis, or treatment. 10.1.031 The methods are useful in the assistance of an animal, a mammal or yet further a human patient. For the purpose of illustration only, a mammal includes but is not limited to a human, a simian, a murine, a bovine, an equine, a porcine or an ovine subject.

This disclosure provides a method of treating a patient having a pre-cancerous oral leukoplakia (LK) lesion or delaying the progression of a pre-cancerous oral LK to oral squamous cell carcinoma, wherein the patient's sample comprising the LK lesion does not have 9p21.3 loss in the sample, the method comprising, or consisting of, or consisting essentially of administering an effective amount of an anti-PD-1 therapy to the patient. In one aspect, the oral LK comprises proliferative verrucous leukoplakia (PVL). 9p21.3 loss intends a partial or full loss of the genetic locus in the cell sample as compared to a patient not having the cancer.

Non-limiting examples of anti-PD-1 treatment is selected from those known in the art, see e.g., those listed in Table 7. In one aspect the treatment, comprises, or consists essentially of, or yet further consists of administration of an effective amount of nivolumab. In a further aspect, the anti-PD-1 treatment comprises, or consists essentially of, or consists of 2 to 6 or 4 doses of nivolumab (about 480 mg IV) every 28 days. In one aspect, the patient is a human patient. When the patient is suffering from cancer, the therapy can be first line, second line, third line or fourth line therapy.

Any appropriate method can be used to measuring the 9p21.3 loss in the patient sample, non-limiting examples of such include a method selected from chromosomal microarrays configured to detect 9p21.3 loss in the sample, target panel sequencing of the sample, sequencing the sample's exome, whole exome sequencing, sequencing the sample's whole genome. In one aspect, the method comprises whole exome sequencing.

In one aspect, the patient is currently suffering from or is in remission from (has previously suffered from) oral squamous cell carcinoma or head and neck cancer. In another aspect, the patient has not been previously diagnosed with OSCC or HNC, or alternatively any prior cancer of any type.

In a further aspect, the patient has or is in remission from (has previously suffered from) a human papilloma virus negative head and neck cancer subject. The cancer can be primary cancer or metastatic.

This disclosure provides a method of treating a patient having a pre-cancerous oral leukoplakia (LK) lesion, comprising, or consisting of, or consisting essentially of measuring for the presence of 9p21.3 loss in sample isolated from the patient (“patient sample”) comprising cells from the oral LK lesion isolated from the patient and administering effective amount of an anti-PD-1 treatment to the patient not a having 9p21.3 loss in their sample, or administering a therapy other than the anti-PD-1 treatment to a patient having 9p21.3 loss in their sample. In one aspect, the oral LK comprises proliferative verrucous leukoplakia (PVL). 9p21.3 loss intends a partial or full loss of the genetic locus in the cell sample as compared to a patient not having the cancer. Non-limiting examples of anti-PD-1 treatment is selected from those known in the art, examples of such are provided in Table 7. In one aspect the treatment, comprises, or consists essentially of, or yet further consists of administration of an effective amount of nivolumab. In a further aspect, the anti-PD-1 treatment comprises, or consists essentially of, or consists of 2 to 6 or 4 doses of nivolumab (about 480 mg IV) every 28 days.

Any appropriate method can be used to measuring the 9p21.3 loss in the patient sample, non-limiting examples of such include a method selected from chromosomal microarrays configured to detect 9p21.3 loss in the sample, target panel sequencing of the sample, sequencing the sample's exome, whole exome sequencing, sequencing the sample's whole genome. In one aspect, the method comprises whole exome sequencing. In one aspect, the patient is a human patient.

In one aspect, the patient is currently suffering from or is in remission from (has previously suffered from) oral squamous cell carcinoma or head and neck cancer. The cancer can be primary cancer or metastatic. In one aspect, the patient is a human patient. When the patient is suffering from cancer, the therapy can be first line, second line, third line or fourth line therapy. It can be combined with other therapies such as tumor resection.

In a further aspect, the patient has or is in remission from (has previously suffered from) a human papilloma virus negative head and neck cancer subject. The cancer can be primary cancer or metastatic.

Also provided is method of delaying the progression of pre-cancerous oral leukoplakia (LK) to oral squamous cell carcinoma in a patient having an oral LK lesion, the method comprising, or consisting essentially of, or yet further consisting of measuring 9p21.3 loss in patient sample comprising cells from the oral LK lesion isolated from the patient; and administering an effective amount of anti-PD-1 treatment to a patient not having 9p21.3 loss in the sample; or administering a therapy other than the anti-PD-1 treatment to a patient having 9p21.3 loss in the sample. In one aspect, the oral LK comprises proliferative verrucous leukoplakia (PVL). 9p21.3 loss intends a partial or full loss of the genetic locus in the cell sample as compared to a patient not having the cancer. Non-limiting examples of anti-PD-1 treatment are known in the art, examples of such are provided in Table 7. In one aspect the treatment, comprises, or consists essentially of, or yet further consists of administration of an effective amount of nivolumab. In a further aspect, the anti-PD-1 treatment comprises, or consists essentially of, or consists of 2 to 6 or 4 doses of nivolumab (about 480 mg IV) every 28 days. In one aspect, the patient is a human patient. When the patient is suffering from cancer, the therapy can be first line, second line, third line or fourth line therapy.

Any appropriate method can be used to measuring the 9p21.3 loss in the patient sample, non-limiting examples of such include a method selected from chromosomal microarrays configured to detect 9p21.3 loss in the sample, target panel sequencing of the sample, sequencing the sample's exome, whole exome sequencing, sequencing the sample's whole genome. In one aspect, the method comprises whole exome sequencing.

In one aspect, the patient is currently suffering from or is in remission from (has previously suffered from) oral squamous cell carcinoma or head and neck cancer. When the patient is suffering from cancer, the therapy can be first line, second line, third line or fourth line therapy.

In a further aspect, the patient has or is in remission from (has previously suffered from) a human papilloma virus negative head and neck cancer subject. The cancer can be primary cancer or metastatic. When the patient is suffering from cancer, the therapy can be first line, second line, third line or fourth line therapy.

Further provided is a method for predicting if a patient having a pre-cancerous oral leukoplakia (LK) lesion will respond to anti-PD-1 therapy, comprising measuring 9p21.3 loss in patient sample comprising cells from the oral LK lesion isolated from the patient, wherein a patient not having a 9p21.3 loss in the sample is likely to respond to anti-PD-1 therapy and a patient having a 9p21.3 loss in the sample is less likely to respond to anti-PD-1 therapy as compared to a patient having the 9p21.3 loss in the sample. In one aspect, the oral LK comprises proliferative verrucous leukoplakia (PVL). 9p21.3 loss intends a partial or full loss of the genetic locus in the cell sample as compared to a patient not having the cancer. Non-limiting examples of anti-PD-1 treatment are known in the art, examples of such are provided in Table 7. In one aspect the method further comprises, or consists essentially of, or consists of administration of an effective amount of nivolumab to the patient not having 9p21.3 loss in the sample. In a further aspect, the anti-PD-1 treatment comprises, or consists essentially of, or consists of 2 to 6 or 4 doses of nivolumab (about 480 mg IV) every 28 days to the patient not having 9p21.3 loss in the sample. In one aspect, the patient is a human patient. When the patient is suffering from cancer, the therapy can be first line, second line, third line or fourth line therapy.

Any appropriate method can be used to measuring the 9p21.3 loss in the patient sample, non-limiting examples of such include a method selected from chromosomal microarrays configured to detect 9p21.3 loss in the sample, target panel sequencing of the sample, sequencing the sample's exome, whole exome sequencing, sequencing the sample's whole genome. In one aspect, the method comprises whole exome sequencing.

In one aspect, the patient is currently suffering from or is in remission from (has previously suffered from) oral squamous cell carcinoma or head and neck cancer. When the patient is suffering from cancer, the therapy can be first line, second line, third line or fourth line therapy.

In a further aspect, the patient has or is in remission from (has previously suffered from) a human papilloma virus negative head and neck cancer subject. The cancer can be primary cancer or metastatic. When the patient is suffering from cancer, the therapy can be first line, second line, third line or fourth line therapy.

### Administration and Dosing

The appropriate amount and dosing regimen of the active agent to be administered to the subject according to any of the methods disclosed herein, is determined by one of ordinary skill in the art. In some embodiments, the active agents, or salts or solvates thereof, is administered to a subject suffering from abnormal cell growth, such as a human, either alone or as part of a pharmaceutically acceptable formulation, once a week, once a day, twice a day, three times a day, or four times a day, or even more frequently.

Administration can be affected by any method that enables delivery of the therapies or compounds to the site of action. These methods include oral routes, intraduodenal routes, parenteral injection (including intravenous, subcutaneous, intramuscular, intravascular or infusion), topical, and rectal administration. Bolus doses can be used, or infusions over a period of 1, 2, 3, 4, 5, 10, 15, 20, 30, 60, 90, 120 or more minutes, or any intermediate time period can also be used, as can infusions lasting 3, 4, 5, 6, 7, 8, 9, 10, 12, 14 16, 20, 24 or more hours or lasting for 1-7 days or more. Infusions can be administered by drip, continuous infusion, infusion pump, metering pump, depot formulation, or any other suitable means.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the subjects to be treated; each unit containing a predetermined quantity of active compound calculated to produce the desired therapeutic effect in association with the required pharmaceutical carrier. The specification for the dosage unit forms of the disclosure are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present disclosure.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens for administration of the chemotherapeutic or immunotherapeutic agent are well-known in the relevant art and would be understood to be encompassed by the skilled artisan once provided the teachings disclosed herein.

### Experimental Examples

The following examples are intended to illustrate but not limit the scope of Applicant's disclosure.

### Patient and Methods

This was an open-label, single-arm phase 2 trial conducted at the Dana-Farber Cancer Institute (DFCI; Boston, MA). Patients with high-risk oral leukoplakia defined by any of the following criteria were eligible: PVL with multifocal (≥2), contiguous ≥3 cm, or a single lesion ≥4 cm in largest diameter (2-3-4 rule) with epithelial dysplasia (any degree); PVL with four quadrant oral cavity involvement; at least one LL with moderate dysplasia, or erythroleukoplakia for which surgery was indicated, but not feasible or the patient refused. Patients were 18 years or older and had an Eastern Cooperative Oncology Group (ECOG) performance status of ≤2. A history of surgically-treated carcinoma in situ (CIS) or early-stage oral squamous cell carcinoma (OSCC) (American Joint Committee on Cancer 2017 8^{th} edition stages I or II) was permitted. The trial was approved by the DF/HCC institutional review board (IRB) (18-387), conducted in accordance with the Declaration of Helsinki and Good Clinical Practice Guidelines, and registered nationally (NCT03692325).

### Treatment

Following written informed consent participants received nivolumab (480 mg IV) on day 1 of a 28-day cycle for 4 cycles. Immunosuppressive medications and doses of corticosteroids >20 mg prednisone equivalent daily were prohibited unless used for immune-related toxicity management.

### PD-L1 IHC

Immunohistochemical quantitative analysis for PD-L1 on baseline epithelial dysplasia tissue samples was obtained using in-house staining (E1L3N clone, Cell Signaling Technology) and reported as a combined positive score (CPS) (range: 0-100) as scored by an expert head and neck pathologist blinded to outcome data. CPS reflected scoring on intralesional epithelial cells of leukoplakia and associated inflammatory cells.

### Tissue and Peripheral Blood Immunoprofiling

Fresh tissues were enzymatically disaggregated in RPMI (Life Technologies)+10% FBS (HyClone), 100 U/ml collagenase type IV (Life Technologies), and 50 μg/ml DNase I (Roche) at 37° C. for 45 minutes and strained through a 40 m filter. The PBMC layers from blood samples were isolated after centrifugation for 10 min at 1000 g. Red blood cells were removed from samples using red blood cell lysis buffer (Biolegend). Cells were incubated with the Live/Dead Zombie NIR (Biolegend) for 5 min in the dark at room temperature. Fc receptors were blocked prior to surface antibody staining using mouse FcR Blocking Reagent (Biolegend). Cells were stained for 15 min on ice in the dark and washed 2× with PBS+2% FBS. Cells were analyzed on a BD LSRFortessa with FACSDiva software (BD Biosciences). Data were analyzed using FlowJo software version 10.7.2. Antibodies were specific for the following human markers: CD69 (FN50), CD38 (HIT2), LAG-3 (11C3C65), TIM-3 (F38-2E2), CD3 (UCHT1), PD-1 (EH12.1), CD45RA (HI100), CTLA-4 (BNI3), CD45 (HI30), CD4 (RPA-T4), CD16 (3G8), CD33 (WM53), PD-L2 (24F.10C12), CD56 (B159), PD-L1 (29E.2A3), CD15 (W6D3), CD31 (WM59), CD19 (HIB19), CD14 (M5E2) from Biolegend; CCR7 (150503), HLA-DR (G46-6) and CD8 (RPA-T8) from Thermo Fisher Scientific.

### Tissue and Peripheral Blood Whole-Exome Sequencing (WES)

Initial processing of pre-treatment epithelial dysplastic tissue samples was performed at the UCSD Biorepository & Tissue Technology Shared Resource Center. Briefly, paraffin was removed from formalin-fixed, paraffin-embedded (FFPE) tissue sections and cores using CitriSolv (Fisher Scientific) followed by ethanol washes, and then tissue was lysed overnight at 56° C. Samples were then incubated at 90° C. to remove DNA crosslinks, and extraction was performed using Qiagen's QIAamp DNA FFPE Tissue Kit. DNA from matching patient peripheral blood mononuclear cells (PBMCs) were extracted using the ALLPREP DNA/RNA Mini Kit. Both FFPE tissue and PBMC DNA were sent to Novogene (Sacramento, CA). DNA whole-exome sequencing library preparation was performed using the IDT xGen Exome Panel v2 kit following the manufacturer's recommendations. Qualified libraries were sequenced on an Illumina platform according to effective concentration and data volume. For FFPE tissue samples, the effective coverage of sequencing was 200× and for PBMC match-normal samples the effective coverage was 100×.

### WES DNA Assembly and Quality Control

Post-sequencing analysis was performed within Triton Shared Compute Cluster (TSCC) at the University of California, San Diego. Briefly, quality assurance of the raw FASTQ files were evaluated using FastQC and Mosdepth [bioinformatics.babraham.ac.uk/projects/fastqc; Pedersen et al., Bioinformatics, 2018, Vol. 34(5): p. 867-8]. Raw sequence reads were aligned to the human reference genome GRCh38. The aligned reads were marked duplicated using MarkDuplicates (Picard)—GATK [McKenna et al., Genome Res, 2010, Vol. 20(9): p. 1297-303]. Concordance between tumor and matched normal samples were evaluated using Conpair [Bergmann et al., Bioinformatics, 2016, Vol. 32(20): p. 3196-8] and only samples with >99.5% concordance were taken forward for subsequent analysis.

### WES Somatic Alteration Identification and Annotation

EnsembleVariantCallingPipeline (EVC) was used to call single nucleotide variants (SNV) and short INDELS. EVC implements the SNV and INDEL variant calling from four variant callers (Mutect2, Strelka2, Varscan2, MuSE) and only passed mutations called with any two variant callers were considered true mutations [McKenna et al., Genome Res, 2010, Vol. 20(9): p. 1297-303; Fan et al., Genome Biol, 2016, Vol. 17(1): p. 178; Kim et al., Nat Methods, 2018, Vol. 15(8): p. 591-4; Koboldt et al., Genome Res, 2012, Vol. 22(3): p. 568-76]. Following annotation of each variant with Ensembl Variant Effect Predictor (VEP, version 106), somatic mutations were taken forward for amino acid change detection [McLaren et al., Genome Biol, 2016, Vol. 17(1): p. 122]. Oncoplot was generated using Maftools [Mayakonda et al., Genome Res, 2018, Vol. 28(11): p. 1747-56]. Possible involvement of driver gene mutations was identified with IntOGen [Gonzalez-Perez et al., Nat Methods, 2013, Vol. 10(11): p. 1081-2]. Copy number alteration detection was performed with ASCAT (v3.1) [Van Loop et al., Proc Natl Acad Sci USA, 2010, Vol. 107(39): p. 16910-5] and focal somatic copy number alterations were detected with GISTIC2.0 [Mermel et al., Genome Biol, 2011, Vol. 12(4): p. R41]. Fisher exact tests were performed within R statistical language [R Core Team (2013) R: A Language and Environment for Statistical Computing. R Foundation for Statistical Computing, V. and www.R-project.org]. Mutational profile and signature analysis were performed with SigProfiler bioinformatic tools developed within the Alexandrov lab [Bergstrom et al., BMC Genomics, 2019, Vol. 20(1): p. 685; S M Ashiqul Islam et al., bioRxiv, 2020, www.doi.org/10.1101/2020.12.13.422570] at UC San Diego. Plotting of figures was performed with Adobe Illustrator and Prism.

### Assessments

Three weeks prior to the first dose of nivolumab and at monthly visits, patients underwent digital intraoral color photography to capture all leukoplakia lesions. Bidimensional measurements were obtained from up to three target lesions (per patient) as determined by one of five oral medicine investigators. Screening and post-treatment biopsies were performed by the same oral medicine investigator for consistency. Fresh tissue biopsies from all target lesions were mandatory at baseline and 30 days after the final dose of nivolumab. Pathologic specimens from each biopsy were examined by two experienced oral pathologists blinded to outcome data (or a third in cases of any scoring discrepancy). New or suspicious non-target lesions or changes in target lesions could trigger re-biopsy at any point.

Response was assessed according to a modified composite scoring system (van der Waal classification)¹⁹ (see FIG. 4). The sum of target lesion point scores (both clinical and pathologic) yielded a composite score. The percent change in composite score before and after treatment determined best overall response. Major response (MR) was a decrease of >80%, partial response (PR) a decrease of 40-80%, stable disease (SD) was neither a MR or PR, and progression of disease (PD) was defined as an increase of ≥10% in the composite score or a CIS or OSCC diagnosis. Patients were followed with clinical exams every 3-4 months until study withdrawal, or up to 5 years.

### Safety

Safety evaluations included laboratory and adverse event (AE) assessments (NCI Common Terminology Criteria version 5.0)20. For patients who developed grade 3 or intolerable grade 2 immune-related (ir)AEs, nivolumab could be interrupted, delayed, or discontinued; certain grade 4 irAEs required discontinuation. AEs were captured up to 3 months after completion of nivolumab.

### Statistical Design

The primary endpoint was best overall response (MR+PR rate) as defined by the percent change in clinical-pathologic composite score. A two-stage Simon optimal design was utilized. When >5 of 33 patients who were eligible and began protocol treatment had disease in response (assuming >1 patient with disease in response among the first 16 patients) there was 84.3% power to rule out a 10% and detect a 25% response rate (using a one-sided exact binomial test, type I error rate of 10%). A response rate of 25% was targeted when considering the cumulative risk of serious irAEs²¹.

Secondary endpoints included safety and CFS defined as the time from trial registration to OSCC or death due to any cause (participants alive without oral cancer were censored at last assessment). Based on studies of PD-L1 expression^{14,22} and somatic 9p21 copy-number loss in advanced OSCC, lung and other tumors^{23,24} we conducted secondary analyses to evaluate the impact of pre-treatment dysplastic tissue PD-L1 expression and 9p21.3 deletion status on outcomes. Exploratory analyses included immunogenomic profiling utilizing multiparametric flow cytometry and whole-exome sequencing (WES).

The primary efficacy population included all eligible patients who began protocol treatment. Response rate was summarized as a proportion with a corresponding two-stage 95% confidence interval (CI). The distribution of CFS was estimated using the Kaplan-Meier method. Logistic regression and Cox proportional hazard models were used to estimate odds ratios (OR) for best overall response and hazard ratios (HR) for CFS, respectively. Fisher's exact test was used to compare somatic copy number alterations (SCNAs) and genomic subsets (two-sided). Wilcoxon signed rank test (paired data) and Wilcoxon rank-sum test (independent) were utilized to analyze both circulating and tissue-based immune profiling parameters (two-sided), employing a Bonferroni-Dunn correction for tests of multiple comparisons. Data as of Sep. 30, 2022 were analyzed.

### Results

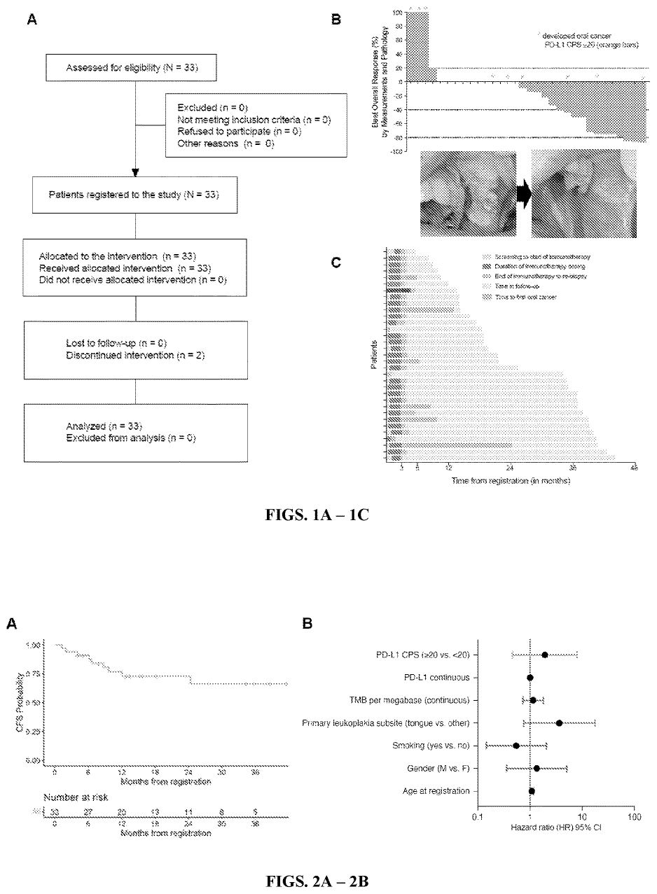

Between Jan. 10, 2019 and Dec. 13, 2021, 33 patients enrolled. All began protocol treatment and are included in analyses (FIG. 1A). Median age was 63 years (range, 32-80) with a slight majority of women (18, 55%), and many were smokers (16, 48%) (Table 1). Eight (24%) had a history of surgically treated early-stage OSCC. Median disease-free interval for those with a head and neck cancer prior to trial entry was 10.5 months (range, 0.3-195). A median of 4 cycles of therapy were received (12% of patients received fewer than all 4 doses).

TABLE 1
Baseline Patient Characteristics

| | Number of | |
|---|---|---|
| Characteristic | patients (%)^{A} N = 33 | |
| Age, years | 63.2 | (32-80) |
| Gender | | |
| Male | 15 | (45) |
| Female | 18 | (55) |
| Race^{B} | | |
| Asian | 1 | (3) |
| White/Caucasian | 31 | (94) |
| Other | 1 | (3) |
| Ethnicity^{B} | | |
| Hispanic | 0 | |
| Non-hispanic | 33 | (100) |
| ECOG performance status | | |
| 0 | 30 | (91) |
| 1 | 3 | (9) |
| Autoimmune history | | |
| Yes | 3 | (9) |
| No | 30 | (91) |
| Smoking history | | |
| Never or ≤10 pack-years | 17 | (52) |
| Former (>10 pack-years) | 15 | (45) |
| Current | 1 | (3) |
| Primary site of disease^{C} | | |
| Oral tongue | 13 | (39) |
| Buccal gingiva | 10 | (30) |
| Palatal gingiva | 1 | (3) |
| Alveolar ridge mucosa | 9 | (27) |
| Number of target lesions | | |
| 1 | 23 | (70) |
| 2 | 7 | (21) |
| 3 | 3 | (9) |
| High-risk oral leukoplakia subtype | | |
| Proliferative verrucous leukoplakia (PVL) | 29 | (88) |
| PVL with 4 quadrant involvement | 2 | (6) |
| Localized leukoplakia with moderate dysplasia | 1 | (3) |
| Erythroleukoplakia | 1 | (3) |
| Worst degree of dysplasia | | |
| identified on biopsy at baseline | | |
| None | 0 | |
| Mild | 24 | (73) |
| Moderate | 8 | (24) |
| Severe or carcinoma in situ (CIS) | 1 | (3) |
| Prior early-stage oral cavity | | |
| squamous cell carcinoma diagnosis | | |
| Yes | 8 | (24) |
| No | 25 | (76) |
| Median doses of nivolumab received (1-4) | 4 | (1-4) |
| First dose of immunotherapy to post- | 115 | (29-171) |

treatment biopsy (in days)

^{A}values are numbers and percentages, except age showing range in parentheses;
^{B}as classified by the participant (‘Other’ denotes mixed race);
^{C}denotes the largest or primary site of oral leukoplakia at trial enrollment as many patients have multifocal sites of involvement in the oral cavity.
Abbreviations: ECOG, Eastern Cooperative Oncology Group; KUS, keratosis of undetermined significance.

Twelve patients (36%) (95% CI, 20.4-54.8) demonstrated a best overall response of MR or PR, with three (9%) demonstrating a >80% reduction in composite score (Table 2).

TABLE 2
Efficiency Measures and Reasons for Treatment Discontinuation

| | Number of patients, | |
|---|---|---|
| Efficacy Measure | N = 33 (%) | |
| Best overall response to immunotherapy^{A} | | |
| Major response | 3 | (9) |
| Partial response | 9 | (27) |
| Stable disease | 16 | (48) |
| Progression of disease^{B} | 4 | (12) |
| Unevaluable^{C} | 1 | (3) |
| Reason for treatment discontinuation | | |
| Completed therapy | 29 | (88) |
| Toxicity to drug | 2 | (6) |
| Progression of disease | 2 | (6) |
| Physician discretion | 0 | |
| Withdrawal of consent | 0 | |
| Death | 0 | |
| Median follow-up (months, range) | 21.1 | (5.4-43.6+) |
| Median cancer-free survival (CFS) | NR | (24.3-NR) |
| (months, 95% CI) | | |
| Number of events^{D} | 9 | (27.3) |
| 1-year CFS (%, 95% CI) | 76.6% | (56.8-88.2) |
| 2-year CFS (%, 95% CI) | 72.8% | (52.6-85.5) |
| 3-year CFS (%, 95% CI) | 66.2% | (43.3-81.5) |

| | |
|---|---|
| Median overall survival (OS) | NR |
| (months, 95% CI) | |
| Number of events | 0 |
| 2-year OS (%, 95% CI) | 100% |

^{A}determined by the change in bidirectional measurements and degree of pathologic dysplasia composite scoring of target leukoplakia lesion(s);
^{B}determined via composite score (at least a 10% increase in the total composite score from baseline) or development of either oral squamous cell carcinoma or carcinoma in situ (CIS) while on study treatment;
^{C}one patient experienced toxicity and withdrew consent from treatment prior to re-biopsy;
^{D}CFS events include development of oral squamous cell carcinoma documented via biopsy confirmation or death whichever occurred first. None of the 9 events were due to death alone.
CI = confidence interval, NR = not reached, “+” = censored at last follow-up as of data cutoff.

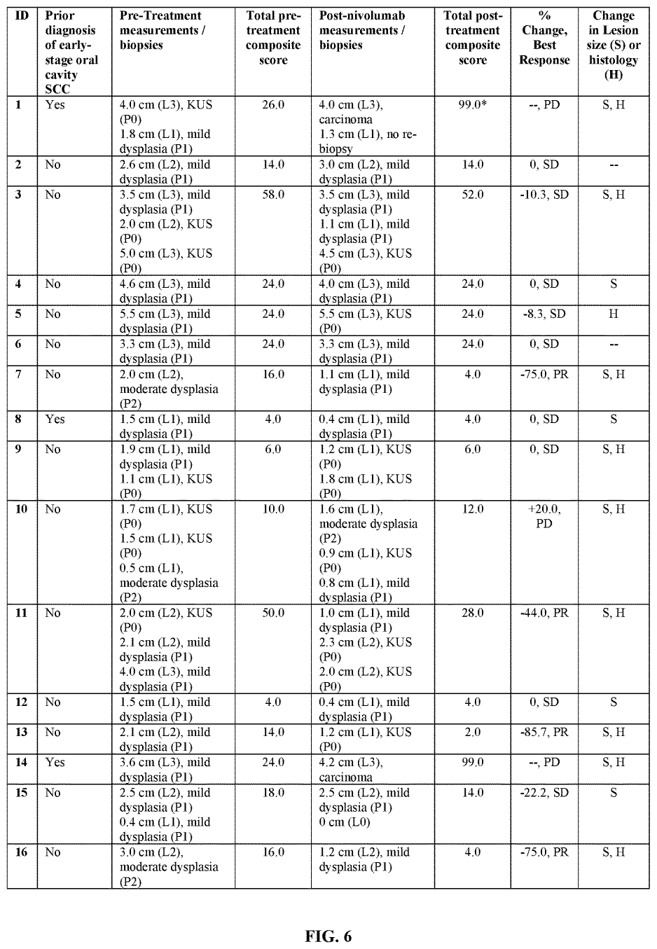

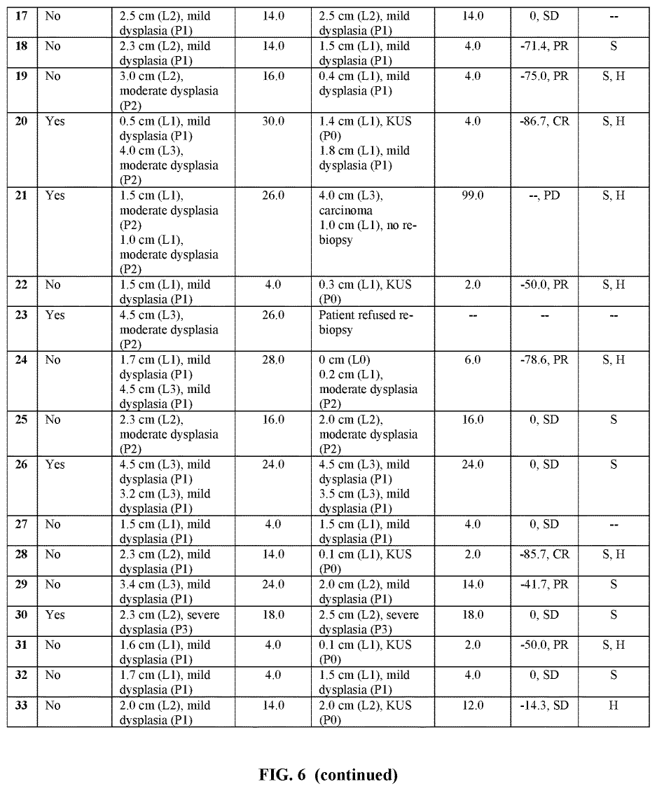

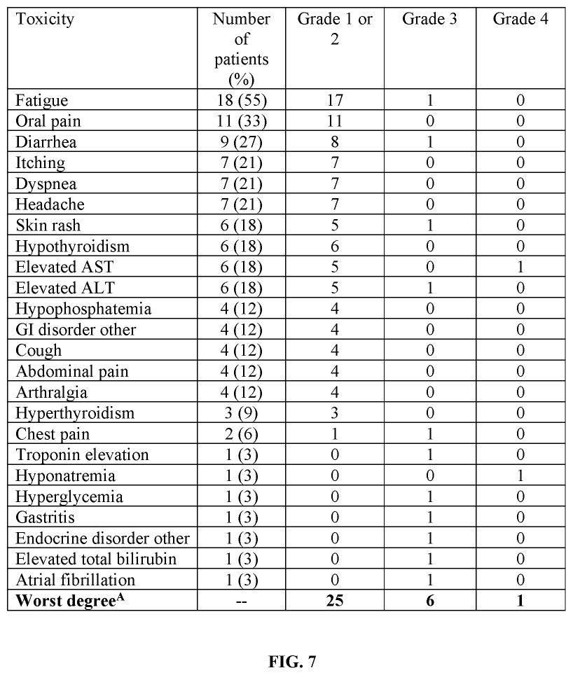

Among individual patients, 2 (6%) had complete resolution of at least one target lesion. Sixteen (48%) had SD and four (12%) patients had a best response of PD (FIGS. 1B-1C). Three of the patients with a best response of PD developed OSCC in a target lesion identified on their end-of-treatment biopsy, the other experienced an increase in the severity of dysplasia in a buccal gingiva target lesion resulting in >20% composite score increase. No patient developed CIS. Six additional patients with a best response other than PD later developed OSCC (Table 3, shown in FIG. 6, Detailed clinical and pathologic assessments of all study patients); of note, 6 of the 9 had a history of early-stage OCSCC. Three of 12 (25%) responders later developed OSCC. Among the 9 patients with an OSCC event, median time from trial registration to a first OSCC event was 6.6 months (range: 1.3-24.3) and median time from the last dose of nivolumab to the development of OSCC was 3.7 months. Eight of nine events were in target lesions.

At a median follow-up of 21.1 months (range: 5.4-43.6), median CFS has not been reached (NR) (95% CI, 24.3 to NR) with a 2-year CFS of 72.8% (95% CI, 52.6-85.5) (FIG. 2). There were 9 CFS events (27.3%) and no deaths. No clinical or pathologic features appeared to impact CFS except a history of early-stage OSCC (HR 13.53, 95% CI 3.3-55.5) (Table 4). The median CFS for patients with a prior oral cavity cancer diagnosis was 1.3 months (95% CI, 6.2-12.1) and for patients without a history of OSCC the median was not reached.

TABLE 4
Association between clinical and pathologic
parameters and cancer-free survival

Total (N = 33)^{A}

| Variable | HR | [95% CI] |
|---|---|---|
| Gender (male vs. female) | 1.34 | 0.36-5.06 |
| Age at registration (continuous)^{B} | 1.08 | 0.99-1.18 |
| Smoking history (yes vs. no) | 0.55 | 0.15-2.05 |
| Primary leukoplakia subsite | 3.64 | 0.76-17.54 |
| (tongue vs. other sites) | | |
| TMB per megabase (continuous)^{B} | 1.15 | 0.73-1.79 |
| PD-L1 CPS (continuous)^{B} | 1.01 | 0.98-1.03 |
| PD-L1 CPS (≥20 vs. <20) | 1.92 | 0.46-8.04 |
| History of prior oral squamous | 13.53 | 3.30-55.52 |
| cell carcinoma (yes vs. no) | | |
| Pre-treatment or (baseline) | 1.04 | 1.00-1.08 |
| composite score (continuous)^{B} | | |

^{A}except TMB where N = 20; ^{B}for every increase in age at registration the risk of CFS event increases by 8%; for every increase in TMB per megabase, the risk of CFS event increases by 15%; for every increase in CPS, the risk of CFS event increases by 1% and for every increase in pre-treatment composite score, the risk of CFS event increases by 4%; HR = hazard ratio (univariate estimates shown), CI-confidence interval, CPS-combined positive score.
Univariate Cox proportional hazard modeling (HR > 1: higher risk of CFS event).
*Categorizing response with an already small sample size reduces power. Linear regression (with post-treatment composite score as the outcome) was used to assess for variability in the pre-treatment (baseline) composite score. First, with pre-treatment PD-L1 CPS alone as primary predictor in the linear regression model: for every 1 unit increase in PD-L1 CPS, post-treatment composite score decreases by 0.06 [post treatment composite score = 13.12-0.06*pre-treatment PD-L1 CPS]; when PD-L1 CPS as the primary clinical predictor adjusted for pre-treatment composite score (all variables are continuous), results are similar: for every 1 unit increase in pre-treatment PD-L1 CPS, post-treatment composite score decreases by 0.11 [post-treatment composite score = 1.05 + 0.73* pre-treatment composite score-0.11*pre-treatment PD-L1 CPS].

Fatigue was the most common AE (18, 55%), followed by oral pain (11, 33%), and diarrhea (9, 27%) (Table 5, shown in FIG. 7, showing adverse events potentially attributable to nivolumab). Seven patients (21.2%) developed grade 3-4 AEs which later resolved. One patient without a cardiac history had atypical chest pain after a half-marathon post-cycle 1 and had an elevated troponin T; cardiology evaluation clarified a low suspicion for immune-related myocarditis, and the patient resumed treatment. Two patients developed immune-related hepatitis. One patient developed immune-related colitis five months post-completion of therapy.

All pre-treatment dysplastic specimens were evaluable for PD-L1 combined positive score (CPS) testing. Scores ranged from 0 to 80 (FIGS. 5A-5B) with 22 (67%) demonstrating a CPS >1. No significant difference was observed in PD-L1 CPS scores among responders vs. non-responders (12.5 vs. 5, p=0.21) and patients with CPS≥20 vs. <20 were not significantly more likely to respond (OR: 4.29, 95% CI, 0.83-25.94) (Table 6).

TABLE 6
Clinical and pathologic predictors of response*

Total (N = 33)^{A}

| Covariate | OR | [95% CI] |
|---|---|---|
| Gender (male vs. female) | 0.45 | 0.10-1.92 |
| Age at registration (continuous)^{B} | 0.99 | 0.92-1.06 |
| Smoking history (yes vs. no) | 1.10 | 0.26-4.64 |
| Primary leukoplakia subsite (tongue | 1.10 | 0.26-4.64 |
| vs. other sites) | | |
| TMB per megabase (continuous)^{B} | 0.83 | 0.44-1.41 |
| PD-L1 CPS (continuous)^{B} | 1.03 | 1.00-1.06 |
| PD-L1 CPS (≥20 vs. <20) | 4.29 | 0.83-25.94 |

^{A}except for TMB where N = 20; ^{B}for every increase in age at registration the odds of being CR/PR decreased by 1%, for every increase in TMB per megabase; the odds of being in CR/PR decrease by 17.1%, and for every increase in CPS; the odds of being in CR/PR increase by 2.8%; OR = odds ratio (univariate estimates shown), CI = confidence interval, CPS = combined positive score; Univariate logistic regression analysis (OR > 1: higher probability of response).
*Categorizing response with an already small sample size reduces power. Linear regression (with post-treatment composite score as the outcome) was used to assess for variability in the pre-treatment (baseline) composite score. First, with pre-treatment PD-L1 CPS alone as primary predictor in the linear regression model: for every 1 unit increase in PD-L1 CPS, post-treatment composite score decreases by 0.06 [post treatment composite score = 13.12-0.06*pre-treatment PD-L1 CPS]; when PD-L1 CPS as the primary clinical predictor adjusted for pre-treatment composite score (all variables are continuous), results are similar: for every 1 unit increase in pre-treatment PD-L1 CPS, post-treatment composite score decreases by 0.11 [post-treatment composite score = 1.05 + 0.73* pre-treatment composite score-0.11*pre-treatment PD-L1 CPS].

Multiparametric flow on paired dysplastic tissue before and after treatment revealed that CD8+ T cells showed greater activation (CD69) and immune checkpoint LAG3 co-expression post-treatment; with increased LAG3 expression among patients with pre-treatment 9p21.3 LOH profiles. Among paired peripheral blood samples, PD-1 expression on both circulating CD4+ and CD8+ T cells decreased significantly (both adjusted p<0.001), while CD38 increased on CD8+ T cells (adjusted p<0.001) (FIG. 5C).

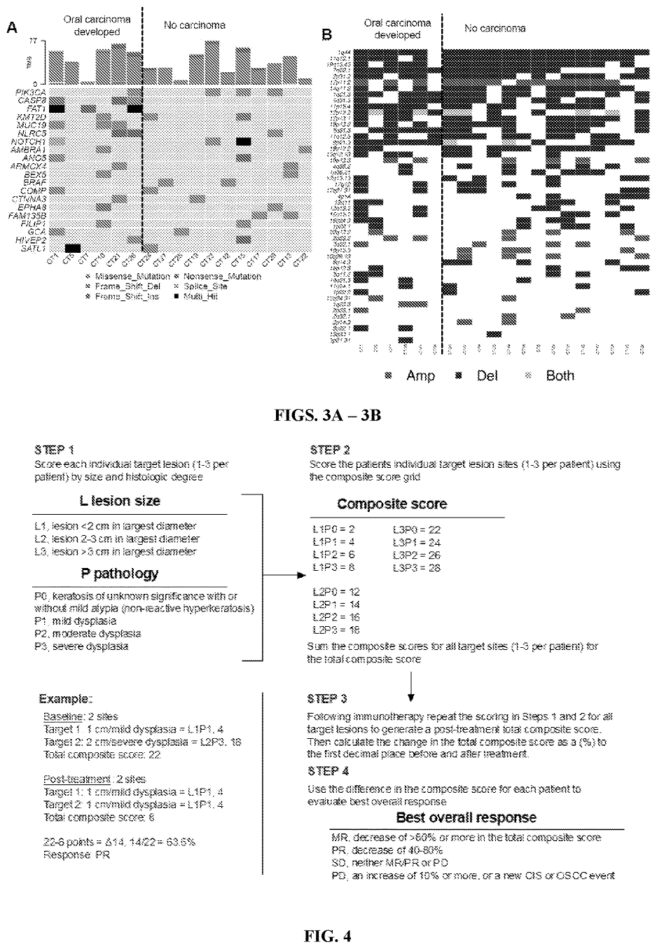

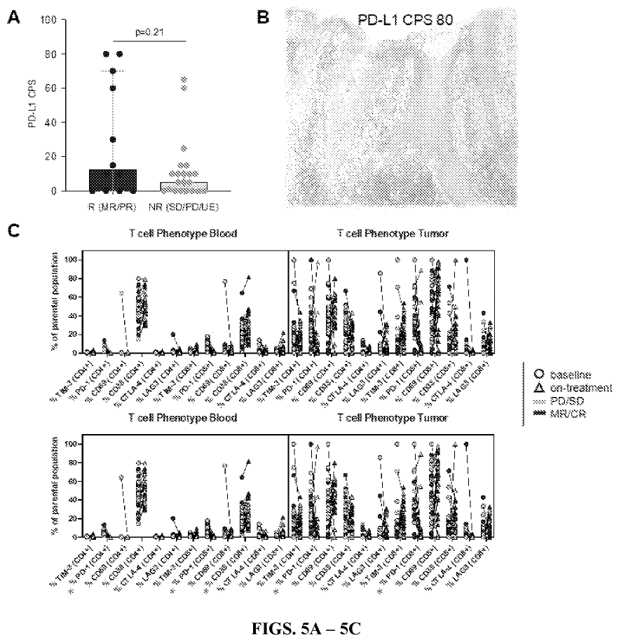

A subset of 23 (70%) patients had adequate tissue for whole exome sequencing (WES). Twenty pairs of paired peripheral blood and oral dysplastic tissue passed quality controls. Pre-treatment median total mutational burden (TMB) was 3.4 mutations per megabase (Mb) (range: 1.4-8.0) and was similar regardless of response (3.6 vs. 2.8, p=0.63), and among those patients who developed cancer vs. not (3.9 vs. 2.9, p=0.51) (FIG. 3A). Genomic driver alterations were similar in patients who developed OSCC vs. not. Missense mutations in PIK3CA were common. SCNAs revealed a range of complex allelic-imbalance profiles; primarily focal deletions, most frequently observed at 1q44 (FIG. 3B). Only 9p21.3 deletion, yielded statistically significant differences between patients who developed OSCC and those who did not. Of 10 patients whose pre-treatment tissue sequencing showed 9p21.3 copy-number loss, 6 (60%) later developed OSCC whereas none of the 10 patients without 9p21.3 loss developed OSCC (p=0.01).

### Discussion

Applicant present the first trial demonstrating the potential efficacy of PD-1 immune checkpoint blockade among patients with high-risk oral precancerous disease. Applicant's data suggest that PD-1 inhibition may yield clinical-pathologic regression in some patients. While some chemoprevention trials have yielded short-term responses to reverse or mute oral carcinogenesis, no therapeutic agents have demonstrated an improvement in CFS and rates of progression to cancer range from 10-30%^{7-9,25-28}.

PVL is an uncommon variant of leukoplakia, occurring in less than 1% of adults, which is aggressive and challenging to treat^{29,30} largely due to non-homogeneous, multifocal lesions, and with the histologic hallmarks being corrugated hyperkeratosis and verrucous hyperplasia with variable dysplasias,^{31,32}. Some degree of dysplasia was required in Applicant's trial with the aim of selecting the highest-risk lesions. Applicant's previous retrospective cohort of PVL patients suggested a 2-year CFS of 82%.¹⁴ In the present trial, Applicant observed a 2-year CFS of 73%, however, Applicant designed the trial with stringent entry criteria, requiring biopsy-proven dysplasia and permitting a history of OSCC. Notably, CFS was a secondary endpoint in Applicant's trial, and the sample size and median follow-up time were limited. It is plausible that Applicant's preliminary CFS rate would have been similar without immunotherapy exposure supporting the need for randomized data. Three responders on trial later developed OSCC, suggesting that Applicant's scoring system and response definitions may not adequately predict CFS. The prognostic impact of tumor size may not be readily generalizable to precancerous lesions, and a one-tier change in histopathology (degree of dysplasia) may not be an optimal outcome measurement. As compared to prior chemoprevention trials Applicant's rate of progression to cancer (27%) was comparable^{7-9,25-28}, while response was defined in prior studies primarily based on lesion size and not histologic change.

Of 9 OSCC events, 6 (67%) were among patients with prior early-stage OSCC with a short median time to failure (<4 months). Including patients with prior cancer events does add some heterogeneity to the trial population, but Applicant felt it was important to include them given their recurrence risk³³. Exclusion of patients with prior oral cancer has been implemented in some chemoprevention studies^{27,28,34,35}, but in the Erlotinib Prevention of Oral Cancer (EPOC) trial⁷, 60% of patients had prior OSCC. That study followed a prevention-adjuvant therapy convergent design³⁶ under the assumption that high-risk patients with oral pre-malignant lesions and resected cancers share molecular alterations for prevention and could be studied in similar settings^{23,37,38}. The cancer events among Applicant's patients were most often pT1 lesions, but structured follow-up may have identified cancers earlier with a bias towards earlier biopsy. Longer follow-up in a larger randomized trial design will be needed to identify a time-to-event or survival benefit. It is unclear whether immunotherapy favorably impacts the pathologic severity of future oral cancer events.

Applicant acknowledges that novel pathologic criteria were required to evaluate efficacy in this first oral precancer ICT prevention trial, as more traditional response criteria would not apply. Applicant chose a modified composite scoring method to quantify response as a function of lesion size and dysplasia across multiple sites, recognizing that analyzing percent changes in composite score can be limited by small sample size and variability in scores. To limit inter-observer variability Applicant required digital intraoral photography with bidimensional measurements, and structured pathologic examination among 2-3 oral pathologists. Applicant recognize that distinguishing mild dysplasia from hyperkeratosis can be subject to interpretation, and most of Applicant's cohort (73%) had mild dysplasia at baseline. Further, Applicant observed a mix of lesion size and/or histologic changes in response to therapy among individual patients. Applicant appreciates that multifocal lesions may have impacted response assessments, but Applicant would not expect spontaneous clinical regression in PVL in the absence of an effective therapy. A time-to-event CFS endpoint may be more generalizable and have broader clinical applicability. It is also worth noting that Applicant's trial population had limited racial and ethnic diversity, and many patients traveled to Applicant's center for treatment which introduces some component of socioeconomic bias. This not only has potential treatment efficacy implications, but also may influence tolerance and affordability.

A major concern for this ICT trial was safety as Applicant treated patients who did not have documented oral cancer. Frequently reported AEs were in line with prior head and neck cancer study populations more broadly¹⁰⁻¹³. Applicant did see some increase in grade 3-4 AEs (21.2%) although Applicant did permit a history of autoimmune disease and all higher grade irAEs resolved in time with no deaths. These findings need to be weighed carefully against the potential for clinical efficacy given concern for a narrow therapeutic risk-benefit ratio.

Genomic studies of precancers have been limited by adequate tissue availability from small biopsies. Therefore, prior studies generally assessed single genes and/or allelic-imbalance using microsatellite markers, detecting 9p21.3 LOH in approximately 45% of PVL patients, depending on the number of markers utilized³⁹. This is the first study employing WES in PVL, which revealed a range of complex SCNA and allelic-imbalance profiles. Recent data from several groups have found that 9p deletions encompassing 9p21 are significant and selective predictors of ICT resistance in advanced OSCC and lung cancer^{23,24,40,41}. This may be due to deletions encompassing the type-I interferon gene cluster⁴², which is often co-deleted with the tumor suppressor CDKN2A, highlighting a key mechanism of immune evasion⁴³. In Applicant's immunogenomic studies, only pre-treatment 9p21.3 deletion yielded statistically significant differences—6/10 patients with 9p21.3 deletion in baseline biopsies later developed cancer. Applicant have previously shown that 9p21.3 copy-number loss is generally a focal event in oral precancer⁴⁴ and associated with an immune-cold signal in OSCC²³ that is enhanced by larger deletions extending to the telomeric band at 9p24.1⁴⁰. Applicant speculate that resistance to the PD-1 inhibitor in this aggressive oral precancerous disease trial may have arisen during ICT resulting from increasing 9p deletion size to encompass 9p24.1, leading to low expression of the therapeutic target (PD-L1) and other immune gene depletion^{23,40,45}. PD-L1 is encoded by gene CD274 which is located on 9p24.1, close to 9p21 and is often co-deleted in advanced HPV-negative HNSCC and lung

Applicant reports the first clinical trial of ICT in patients with precancerous disease, specifically patients with high-risk oral precancer to mitigate progression to OSCC. This trial met its primary response endpoint but few patients had complete lesion regression. Other studies using immunotherapy to treat patients with high-risk oral premalignant lesions are ongoing (NCT03603223, NCT04504552). Recognizing the limitations and complexity of measuring treatment effects in precancer trials, for the first time Applicant demonstrates potential efficacy and acceptable safety with the use of ICT in a high-risk precancerous population. A next step would be to consider a larger, randomized controlled precision immunotherapy trial favoring CFS as a primary outcome and stratified by prior history of early-stage treated OSCC and 9p21.3 loss.

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### REFERENCES

1. Mello F W, Miguel A F P, Dutra K L, Porporatti A L, Warnakulasuriya S, Guerra E N S, Rivero E R C. Prevalence of oral potentially malignant disorders: A systematic review and meta-analysis. J Oral Pathol Med. 2018 August; 47(7):633-640. doi: 10.1111/jop.12726. Epub 2018 Jun. 6. PMID: 29738071.
2. Chaturvedi A K, Udaltsova N, Engels E A, Katzel J A, Yanik E L, Katki H A, Lingen M W, Silverberg M J. Oral Leukoplakia and Risk of Progression to Oral Cancer: A Population-Based Cohort Study. J Natl Cancer Inst. 2020 Oct. 1; 112(10):1047-1054. doi: 10.1093/jnci/djz238. PMID: 31860085; PMCID: PMC7566351.
3. Speight P M, Khurram S A, Kujan O. Oral potentially malignant disorders: risk of progression to malignancy. Oral Surg Oral Med Oral Pathol Oral Radiol. 2018 June; 125(6):612-627. doi: 10.1016/j.oooo.2017.12.011. Epub 2017 Dec. 29. PMID: 29396319.
4. Torrejon-Moya A, Jane-Salas E, López-López J. Clinical manifestations of oral proliferative verrucous leukoplakia: A systematic review. J Oral Pathol Med. 2020 May; 49(5):404-408. doi: 10.1111/jop.12999. Epub 2020 Feb. 9. PMID: 31990082.
5. Villa A, Menon R S, Kerr A R, De Abreu Alves F, Guollo A, Ojeda D, Woo S B. Proliferative leukoplakia: Proposed new clinical diagnostic criteria. Oral Dis. 2018 July; 24(5):749-760. doi: 10.1111/odi.12830. Epub 2018 May 2. PMID: 29337414.
6. Archibald H, Buryska S, Ondrey F G. An active surveillance program in oral preneoplasia and translational oncology benefit. Laryngoscope Investig Otolaryngol. 2021 Jul. 21; 6(4):764-772. doi: 10.1002/lio2.612. PMID: 34401501; PMCID: PMC8356884.
7. William W N Jr, Papadimitrakopoulou V, Lee J J, Mao L, Cohen E E, Lin H Y, Gillenwater A M, Martin J W, Lingen M W, Boyle J O, Shin D M, Vigneswaran N, Shinn N, Heymach J V, Wistuba I I, Tang X, Kim E S, Saintigny P, Blair E A, Meiller T, Gutkind J S, Myers J, El-Naggar A, Lippman S M. Erlotinib and the Risk of Oral Cancer: The Erlotinib Prevention of Oral Cancer (EPOC) Randomized Clinical Trial. JAMA Oncol. 2016 February; 2(2):209-16. doi: 10.1001/jamaoncol.2015.4364. PMID: 26540028; PMCID: PMC4771491.
8. Lippman S M, Batsakis J G, Toth B B, Weber R S, Lee J J, Martin J W, Hays G L, Goepfert H, Hong W K. Comparison of low-dose isotretinoin with beta carotene to prevent oral carcinogenesis. N Engl J Med. 1993 Jan. 7; 328(1):15-20. doi: 10.1056/NEJM199301073280103. PMID: 8416267.
9. Papadimitrakopoulou V A, William W N Jr, Dannenberg A J, Lippman S M, Lee J J, Ondrey F G, Peterson D E, Feng L, Atwell A, El-Naggar A K, Nathan C O, Helman J I, Du B, Yueh B, Boyle J O. Pilot randomized phase II study of celecoxib in oral premalignant lesions. Clin Cancer Res. 2008 Apr. 1; 14(7):2095-101. doi: 10.1158/1078-0432.CCR-07-4024. PMID: 18381950.
10. Cohen E E W, Soulieres D, Le Tourneau C, Dinis J, Licitra L, Ahn M J, Soria A, Machiels J P, Mach N, Mehra R, Burtness B, Zhang P, Cheng J, Swaby R F, Harrington K J; KEYNOTE-040 investigators. Pembrolizumab versus methotrexate, docetaxel, or cetuximab for recurrent or metastatic head-and-neck squamous cell carcinoma (KEYNOTE-040): a randomised, open-label, phase 3 study. Lancet. 2019 Jan. 12; 393(10167):156-167. doi: 10.1016/S0140-6736(18)31999-8. Epub 2018 Nov. 30. Erratum in: Lancet. 2019 Jan. 12; 393(10167):132. PMID: 30509740.
11. Burtness B, H. K., Greil R, Soulieres D, Tahara M, de Castro G Jr, Psyrri A, Baste N, Neupane P, Bratland A, Fuereder T, Hughes BGM, Mesia R, Ngamphaiboon N, Rordorf T, Wan Ishak W Z, Hong R L, Gonzilez Mendoza R, Roy A, Zhang Y, Gumuscu B, Cheng J D, Jin F, Rischin D; KEYNOTE-048 Investigators, Pembrolizumab alone or with chemotherapy versus cetuximab with chemotherapy for recurrent or metastatic squamous cell carcinoma of the head and neck (KEYNOTE-048): a randomised, open-label, phase 3 study. Lancet, 2019. 394(10212): p. 1915-28.
12. Ferris R L, Blumenschein G Jr, Fayette J, Guigay J, Colevas A D, Licitra L, Harrington K, Kasper S, Vokes E E, Even C, Worden F, Saba N F, Iglesias Docampo L C, Haddad R, Rordorf T, Kiyota N, Tahara M, Monga M, Lynch M, Geese W J, Kopit J, Shaw J W, Gillison M L. Nivolumab for Recurrent Squamous-Cell Carcinoma of the Head and Neck. N Engl J Med. 2016 Nov. 10; 375(19):1856-1867. doi: 10.1056/NEJMoa1602252. Epub 2016 Oct. 8. PMID: 27718784; PMCID: PMC5564292.
13. Seiwert T Y, Burtness B, Mehra R, Weiss J, Berger R, Eder J P, Heath K, McClanahan T, Lunceford J, Gause C, Cheng J D, Chow L Q. Safety and clinical activity of pembrolizumab for treatment of recurrent or metastatic squamous cell carcinoma of the head and neck (KEYNOTE-012): an open-label, multicentre, phase 1b trial. Lancet Oncol. 2016 July; 17(7):956-965. doi: 10.1016/S1470-2045(16)30066-3. Epub 2016 May 27. PMID: 27247226.
14. Hanna G J, Villa A, Mistry N, Jia Y, Quinn C T, Turner M M, Felt K D, Pfaff K, Haddad R I, Uppaluri R, Rodig S J, Woo S B, Egloff A M, Hodi F S. Comprehensive Immunoprofiling of High-Risk Oral Proliferative and Localized Leukoplakia. Cancer Res Commun. 2021 Oct. 13; 1(1):30-40. doi: 10.1158/2767-9764.CRC-21-0060. Erratum in: Cancer Res Commun. 2022 May 19; 2(5):390. PMID: 36860910; PMCID: PMC9973379.
15. Spira A, Disis M L, Schiller J T, Vilar E, Rebbeck T R, Bejar R, Ideker T, Arts J, Yurgelun M B, Mesirov J P, Rao A, Garber J, Jaffee E M, Lippman S M. Leveraging premalignant biology for immune-based cancer prevention. Proc Natl Acad Sci USA. 2016 Sep. 27; 113(39):10750-8. doi: 10.1073/pnas.1608077113. Epub 2016 Sep. 16. PMID: 27638202; PMCID: PMC5047191.
16. Mascaux C, Angelova M, Vasaturo A, Beane J, Hijazi K, Anthoine G, Buttard B, Rothe F, Willard-Gallo K, Haller A, Ninane V, Burny A, Sculier J P, Spira A, Galon J. Immune evasion before tumour invasion in early lung squamous carcinogenesis. Nature. 2019 July; 571(7766):570-575. doi: 10.1038/s41586-019-1330-0. Epub 2019 Jun. 26. PMID: 31243362.
17. Pennycuick A, Teixeira V H, AbdulJabbar K, Raza SEA, Lund T, Akarca A U, Rosenthal R, Kalinke L, Chandrasekharan D P, Pipinikas C P, Lee-Six H, Hynds R E, Gowers KHC, Henry J Y, Millar F R, Hagos Y B, Denais C, Falzon M, Moore D A, Antoniou S, Durrenberger P F, Furness A J, Carroll B, Marceaux C, Asselin-Labat M L, Larson W, Betts C, Coussens L M, Thakrar R M, George J, Swanton C, Thirlwell C, Campbell P J, Marafioti T, Yuan Y, Quezada S A, McGranahan N, Janes S M. Immune Surveillance in Clinical Regression of Preinvasive Squamous Cell Lung Cancer. Cancer Discov. 2020 October; 10(10):1489-1499. doi: 10.1158/2159-8290.CD-19-1366. Epub 2020 Jul. 20. PMID: 32690541; PMCID: PMC7611527.
18. Krysan K, Tran L M, Dubinett S M. Immunosurveillance and Regression in the Context of Squamous Pulmonary Premalignancy. Cancer Discov. 2020 October; 10(10):1442-1444. doi: 10.1158/2159-8290.CD-20-1087. PMID: 33004476; PMCID: PMC8396076.
19. van der Waal I, Schepman K P, van der Meij E H. A modified classification and staging system for oral leukoplakia. Oral Oncol. 2000 May; 36(3):264-6. doi: 10.1016/s1368-8375(99)00092-5. PMID: 10793328.
20. U S Department of Health and Human Services, N., Common Terminology Criteria for Adverse Events (CTCAE) Version 5.0. 2017.
21. Magee D E, Hird A E, Klaassen Z, Sridhar S S, Nam R K, Wallis C J D, Kulkarni G S. Adverse event profile for immunotherapy agents compared with chemotherapy in solid organ tumors: a systematic review and meta-analysis of randomized clinical trials. Ann Oncol. 2020 January; 31(1):50-60. doi: 10.1016/j.annonc.2019.10.008. PMID: 31912796.
22. Cohen E E W, Bell R B, Bifulco C B, Burtness B, Gillison M L, Harrington K J, Le Q T, Lee N Y, Leidner R, Lewis R L, Licitra L, Mehanna H, Mell L K, Raben A, Sikora A G, Uppaluri R, Whitworth F, Zandberg D P, Ferris R L. The Society for Immunotherapy of Cancer consensus statement on immunotherapy for the treatment of squamous cell carcinoma of the head and neck (HNSCC). J Immunother Cancer. 2019 Jul. 15; 7(1):184. doi: 10.1186/s40425-019-0662-5. PMID: 31307547; PMCID: PMC6632213
23. William W N Jr, Zhao X, Bianchi J J, Lin H Y, Cheng P, Lee J J, Carter H, Alexandrov L B, Abraham J P, Spetzler D B, Dubinett S M, Cleveland D W, Cavenee W, Davoli T, Lippman S M. Immune evasion in HPV-head and neck precancer-cancer transition is driven by an aneuploid switch involving chromosome 9p loss. Proc Natl Acad Sci USA. 2021 May 11; 118(19):e2022655118. doi: 10.1073/pnas.2022655118. PMID: 33952700; PMCID: PMC8126856.
24. Han G, Yang G, Hao D, Lu Y, Thein K, Simpson B S, Chen J, Sun R, Alhalabi O, Wang R, Dang M, Dai E, Zhang S, Nie F, Zhao S, Guo C, Hamza A, Czerniak B, Cheng C, Siefker-Radtke A, Bhat K, Futreal A, Peng G, Wargo J, Peng W, Kadara H, Ajani J, Swanton C, Litchfield K, Ahnert J R, Gao J, Wang L. 9p21 loss confers a cold tumor immune microenvironment and primary resistance to immune checkpoint therapy. Nat Commun. 2021 Sep. 23; 12(1):5606. doi: 10.1038/s41467-021-25894-9. PMID: 34556668; PMCID: PMC8460828.
25. Saba N F, Haigentz M Jr, Vermorken J B, Strojan P, Bossi P, Rinaldo A, Takes R P, Ferlito A. Prevention of head and neck squamous cell carcinoma: removing the “chemo” from “chemoprevention”. Oral Oncol. 2015 February; 51(2):112-8. doi: 10.1016/j.oraloncology.2014.11.002. Epub 2014 Nov. 26. PMID: 25434586.
26. Rudin C M, Cohen E E, Papadimitrakopoulou V A, Silverman S Jr, Recant W, El-Naggar A K, Stenson K, Lippman S M, Hong W K, Vokes E E. An attenuated adenovirus, ONYX-015, as mouthwash therapy for premalignant oral dysplasia. J Clin Oncol. 2003 Dec. 15; 21(24):4546-52. doi: 10.1200/JCO.2003.03.544. Epub 2003 Nov. 3. PMID: 14597742; PMCID: PMC4523388.
27. Papadimitrakopoulou V A, Lee J J, William W N Jr, Martin J W, Thomas M, Kim E S, Khuri F R, Shin D M, Feng L, Hong W K, Lippman S M. Randomized trial of 13-cis retinoic acid compared with retinyl palmitate with or without beta-carotene in oral premalignancy. J Clin Oncol. 2009 Feb. 1; 27(4):599-604. doi: 10.1200/JCO.2008.17.1850. Epub 2008 Dec. 15. PMID: 19075276; PMCID: PMC2645856.
28. Mulshine J L, Atkinson J C, Greer R O, Papadimitrakopoulou V A, Van Waes C, Rudy S, Martin J W, Steinberg S M, Liewehr D J, Avis I, Linnoila R I, Hewitt S, Lippman S M, Frye R, Cavanaugh P F Jr. Randomized, double-blind, placebo-controlled phase IIb trial of the cyclooxygenase inhibitor ketorolac as an oral rinse in oropharyngeal leukoplakia. Clin Cancer Res. 2004 Mar. 1; 10(5):1565-73. doi: 10.1158/1078-0432.ccr-1020-3. PMID: 15014005.
29. Abadie W M, Partington E J, Fowler C B, Schmalbach C E. Optimal Management of Proliferative Verrucous Leukoplakia: A Systematic Review of the Literature. Otolaryngol Head Neck Surg. 2015 October; 153(4):504-11. doi: 10.1177/0194599815586779. Epub 2015 Jun. 4. PMID: 26044786.
30. Pentenero M, Meleti M, Vescovi P, Gandolfo S. Oral proliferative verrucous leucoplakia: are there particular features for such an ambiguous entity?A systematic review. Br J Dermatol. 2014 May; 170(5):1039-47. doi: 10.1111/bjd.12853. PMID: 24471527.
31. Alabdulaaly L, Villa A, Chen T, Kerr A, Ross N, Abreu Alves F, Guollo A, Woo S B. Characterization of initial/early histologic features of proliferative leukoplakia and correlation with malignant transformation: a multicenter study. Mod Pathol. 2022 August; 35(8):1034-1044. doi: 10.1038/s41379-022-01021-x. Epub 2022 Feb. 19. PMID: 35184151.
32. Thompson L D R, Fitzpatrick S G, Müller S, Eisenberg E, Upadhyaya J D, Lingen M W, Vigneswaran N, Woo S B, Bhattacharyya I, Bilodeau E A, Carlos R, Islam M N, Leon M E, Lewis J S Jr, Magliocca K R, Mani H, Mehrad M, Purgina B, Richardson M, Wenig B M, Cohen D M. Proliferative Verrucous Leukoplakia: An Expert Consensus Guideline for Standardized Assessment and Reporting. Head Neck Pathol. 2021 June; 15(2):572-587. doi: 10.1007/s12105-020-01262-9. Epub 2021 Jan. 7. PMID: 33415517; PMCID: PMC8134585.
33. Ren Z H, Xu J L, Li B, Fan T F, Ji T, Zhang C P. Elective versus therapeutic neck dissection in node-negative oral cancer: Evidence from five randomized controlled trials. Oral Oncol. 2015 November; 51(11):976-981. doi: 10.1016/j.oraloncology.2015.08.009. Epub 2015 Aug. 28. PMID: 26321080.
34. Chiesa F, Tradati N, Marazza M, Rossi N, Boracchi P, Mariani L, Clerici M, Formelli F, Barzan L, Carrassi A, et al. Prevention of local relapses and new localisations of oral leukoplakias with the synthetic retinoid fenretinide (4-HPR). Preliminary results. Eur J Cancer B Oral Oncol. 1992 October; 28B(2):97-102. doi: 10.1016/0964-1955(92)90035-y. PMID: 1306735.
35. Kuriakose M A, Ramdas K, Dey B, Iyer S, Rajan G, Elango K K, Suresh A, Ravindran D, Kumar R R, R P, Ramachandran S, Kumar N A, Thomas G, Somanathan T, Ravindran H K, Ranganathan K, Katakam S B, Parashuram S, Jayaprakash V, Pillai M R. A Randomized Double-Blind Placebo-Controlled Phase IIB Trial of Curcumin in Oral Leukoplakia. Cancer Prev Res (Phila). 2016 August; 9(8):683-91. doi: 10.1158/1940-6207.CAPR-15-0390. Epub 2016 Jun. 7. PMID: 27267893.
36. Abbruzzese J L, Lippman S M. The convergence of cancer prevention and therapy in early-phase clinical drug development. Cancer Cell. 2004 October; 6(4):321-6. doi: 10.1016/j.ccr.2004.09.021. PMID: 15488755.
37. Teixeira V H, Pipinikas C P, Pennycuick A, Lee-Six H, Chandrasekharan D, Beane J, Morris T J, Karpathakis A, Feber A, Breeze C E, Ntolios P, Hynds R E, Falzon M, Capitanio A, Carroll B, Durrenberger P F, Hardavella G, Brown J M, Lynch A G, Farmery H, Paul D S, Chambers R C, McGranahan N, Navani N, Thakrar R M, Swanton C, Beck S, George P J, Spira A, Campbell P J, Thirlwell C, Janes S M. Deciphering the genomic, epigenomic, and transcriptomic landscapes of pre-invasive lung cancer lesions. Nat Med. 2019 March; 25(3):517-525. doi: 10.1038/s41591-018-0323-0. Epub 2019 Jan. 21. PMID: 30664780.
38. Spira A, Yurgelun M B, Alexandrov L, Rao A, Bejar R, Polyak K, Giannakis M, Shilatifard A, Finn O J, Dhodapkar M, Kay N E, Braggio E, Vilar E, Mazzilli S A, Rebbeck T R, Garber J E, Velculescu V E, Disis M L, Wallace D C, Lippman S M. Precancer Atlas to Drive Precision Prevention Trials. Cancer Res. 2017 Apr. 1; 77(7):1510-1541. doi: 10.1158/0008-5472.CAN-16-2346. PMID: 28373404; PMCID: PMC6681830
39. Kresty L A, Mallery S R, Knobloch T J, Li J, Lloyd M, Casto B C, Weghorst C M. Frequent alterations of p16INK4a and p14ARF in oral proliferative verrucous leukoplakia. Cancer Epidemiol Biomarkers Prev. 2008 November; 17(11):3179-87. doi: 10.1158/1055-9965.EPI-08-0574. PMID: 18990760.
40. Zhao X, Cohen EEW, William W N Jr, Bianchi J J, Abraham J P, Magee D, Spetzler D B, Gutkind J S, Alexandrov L B, Cavenee W K, Lippman S M, Davoli T. Somatic 9p24.1 alterations in HPV-head and neck squamous cancer dictate immune microenvironment and anti-PD-1 checkpoint inhibitor activity. Proc Natl Acad Sci USA. 2022 Nov. 22; 119(47):e2213835119. doi: 10.1073/pnas.2213835119. Epub 2022 Nov. 17. PMID: 36395141; PMCID: PMC9704728
41. Ebot E M, Duncan D L, Tolba K, Fabrizio D, Frampton G M, Comment L A, Albacker L A. Deletions on 9p21 are associated with worse outcomes after anti-PD-1/P D-L1 monotherapy but not chemoimmunotherapy. NPJ Precis Oncol. 2022 Jun. 23; 6(1):44. doi: 10.1038/s41698-022-00286-4. PMID: 35739333; PMCID: PMC9225995.
42. Barriga F M, Tsanov K M, Ho Y J, Sohail N, Zhang A, Baslan T, Wuest A N, Del Priore I, Meškauskaité B, Livshits G, Alonso-Curbelo D, Simon J, Chaves-Perez A, Bar-Sagi D, Iacobuzio-Donahue C A, Notta F, Chaligne R, Sharma R, Pe'er D, Lowe S W. MACHETE identifies interferon-encompassing chromosome 9p21.3 deletions as mediators of immune evasion and metastasis. Nat Cancer. 2022 November; 3(11):1367-1385. doi: 10.1038/s43018-022-00443-5. Epub 2022 Nov. 7. PMID: 36344707; PMCID: PMC9701143.
43. Thol K, Pawlik P, McGranahan N. Therapy sculpts the complex interplay between cancer and the immune system during tumour evolution. Genome Med. 2022 Dec. 7; 14(1):137. doi: 10.1186/si3073-022-01138-3. PMID: 36476325; PMCID: PMC9730559
44. William W N Jr, Zhang J, Zhao X, Parra E R, Uraoka N, Lin H Y, Peng S A, El-Naggar A K, Rodriguez-Canales J, Song J, Gillenwater A M, Wistuba I I, Myers J N, Gold K A, Ferrarotto R, Hwu P, Davoli T, Lee J J, Heymach J V, Papadimitrakopoulou V A, Lippman S M. Spatial P D-L1, immune-cell microenvironment, and genomic copy-number alteration patterns and drivers of invasive-disease transition in prospective oral precancer cohort. Cancer. 2023 Mar. 1; 129(5):714-727. doi: 10.1002/cncr.34607. Epub 2023 Jan. 3. PMID: 36597662.
45. Saloura V, Izumchenko E, Zuo Z, Bao R, Korzinkin M, Ozerov I, Zhavoronkov A, Sidransky D, Bedi A, Hoque M O, Koeppen H, Keck M K, Khattri A, London N, Kotlov N, Fatima A, Vougiouklakis T, Nakamura Y, Lingen M, Agrawal N, Savage P A, Kron S, Kline J, Kowanetz M, Seiwert T Y. Immune profiles in primary squamous cell carcinoma of the head and neck. Oral Oncol. 2019 September; 96:77-88. doi: 10.1016/j.oraloncology.2019.06.032. Epub 2019 Jul. 12. PMID: 31422218; PMCID: PMC7893610.

1. A method of treating a patient having a pre-cancerous oral leukoplakia (LK) lesion or delaying the progression of a pre-cancerous oral LK to oral squamous cell carcinoma, wherein the patient's sample comprising the LK lesion does not have 9p21.3 loss in the sample, the method comprising administering an effective amount of an anti-PD-1 or anti-PD-L1 treatment to the patient, and optionally wherein the oral LK comprises a proliferative verrucous leukoplakia (PVL).

2. The method of claim 1, wherein the 9p21.3 loss is measured by a method selected from chromosomal microarrays configured to detect 9p21.3 loss in the sample, target panel sequencing of the sample, sequencing the sample's exome, sequencing the sample's whole genome, or whole exome sequencing.

3. The method of claim 1, wherein the patient has or has suffered from oral squamous cell carcinoma or head and neck cancer.

4. The method of claim 1, wherein the patient has or has suffered from a human papilloma virus negative head and neck cancer subject.

5. The method of claim 1, wherein the patient is a human patient.

6. The method of claim 1, wherein the anti-PD-1 or anti-PD-L1 treatment is selected from a therapy identified in Table 7.

7. The method of claim 1, wherein the anti-PD-1 treatment comprises administration of nivolumab.

8. The method of claim 1, wherein the anti-PD-1 treatment comprises 2 to 6 or 4 doses of nivolumab (about 480 mg IV) every 28 days.

9. A method of treating pre-cancerous oral leukoplakia (LK) or delaying the progression of pre-cancerous oral leukoplakia (LK) to oral squamous cell carcinoma in a patient having an oral LK lesion, optionally wherein the oral LK comprises a proliferative verrucous leukoplakia (PVL), the method comprising:
(a) measuring 9p21.3 loss in patient sample comprising cells from the oral LK lesion isolated from the patient; and
(b) administering an effective amount of anti-PD-1 anti-PD-L1 treatment to a patient not having 9p21.3 loss in the sample; or
(c) administering effective amount of a therapy other than the anti-PD-1 or anti-PD-L1 treatment to a patient having 9p21.3 loss in the sample.

10. The method of claim 9, wherein the measuring 9p21.3 loss of the sample comprises a method selected from chromosomal microarrays configured to detect 9p21.3 loss in the sample, target panel sequencing of the sample, sequencing the sample's exome, sequencing the sample's whole genome, or whole exome sequencing.

11. The method of claim 9, wherein the patient has or has suffered from oral squamous cell carcinoma or head and neck cancer.

12. The method of claim 9, wherein the patient has or has suffered from a human papilloma virus negative head and neck cancer subject.

13. The method of any of claim 9, wherein the patient is a human patient.

14. The method of claim 9, wherein the anti-PD-1 anti-PD-L1 treatment is selected from a therapy identified in Table 7.

15. The method of claim 9, wherein the anti-PD-1 treatment comprises administration of nivolumab.

16. The method of any of claim 9, wherein the anti-PD-1 treatment comprises 2 to 6 or 4 doses of nivolumab (about 480 mg IV) every 28 days.

17. A method of for predicting if a having a pre-cancerous oral leukoplakia (LK) lesion, will respond to anti-PD-1 therapy, comprising measuring 9p21.3 loss in patient sample comprising cells from the oral LK lesion isolated from the patient, wherein a patient not having a 9p21.3 loss in the sample is likely to respond to anti-PD-1 therapy and a patient having a 9p21.3 loss in the sample is less likely to respond to anti-PD-1 therapy as compared to a patient having the 9p21.3 loss in the sample.

18. The method of claim 17, wherein the oral LK comprises a proliferative verrucous leukoplakia (PVL).

19. The method of claim 17, wherein the measuring 9p21.3 loss of the sample comprises a method selected from chromosomal microarrays configured to detect 9p21.3 loss in the sample, target panel sequencing of the sample, sequencing the sample's exome, sequencing the sample's whole genome, or whole exome sequencing.

20. The method of claim 17, wherein the patient has or has suffered from oral squamous cell carcinoma or head and neck cancer.
